(19) United States
(12) Patent Application Publication
(10) Pub. No.: US 2011/0125075 A1
(43) Pub. Date: May 26, 2011

(54) METHOD FOR CONTROL OF ELECTROPORATION APPARATUS

(75) Inventors: Yoshifumi Takei, Aichi (JP); Kenji Kadomatsu, Aichi (JP); Tatsuya Fujishima, Aichi (JP); Takashi Muramatsu, Aichi (JP)

(73) Assignee: National University Corporation Nagoya University, Nagoya-shi (JP)

(21) Appl. No.: 11/988,832

(22) PCT Filed: Jul. 14, 2006
(86) PCT No.: PCT/JP2006/314059

§ 371 Date: Nov. 10, 2008

(30) Foreign Application Priority Data
Jul. 15, 2005 (JP) 2005-207067

(51) Int. Cl. A61N 1/30; A01K 67/027; C12Q 1/02

(52) U.S. Cl. 604/20; 800/25; 800/3; 435/29

(57) ABSTRACT

A method for controlling an electroporation apparatus for use in an animal such as human and a non-human animal, the method comprising a step of applying a voltage to an electrode of the electroporation apparatus placed in/on a biological sample of the animal in the presence of a nucleic acid construct capable of inhibiting the expression of a gene in the animal. In this manner, a nucleic acid construct can be introduced into a cell of a living body with good efficiency.

### TECHNICAL FIELD

The present invention relates to technology of transfecting a nucleic acid construct capable of inhibiting gene expression into an organism by electroporation and to the utilization thereof.

### BACKGROUND ART

There have been attempts in recent years to inhibit the expression of a particular gene within a cell by the transfection into the cell of a nucleic acid construct that brings about an inhibition of gene expression by RNA interference. It is anticipated that the RNA interference-mediated inhibition of gene expression will be applied to the prevention and treatment of various diseases, and investigations are underway into, for example, stabilization of the RNA and effective techniques for transfecting the pertinent nucleic acid into organisms.

For example, a method of delivery into a target tissue using atelocollagen has already been disclosed (Y. TAKEI et al., Cancer Research, 64, 3365-3370, May 15, 2004). A high anti-tumor effect was observed in mice using this delivery method. Atelocollagen is believed to raise the cell uptake efficiency and to stabilize siRNA due to its administration into tissue in a mode in which it has formed a complex with the siRNA.

### DISCLOSURE OF THE INVENTION

The transfection efficiency into cells is of utmost importance in the expression of RAN interference by a nucleic acid construct, for example, siRNA. Atelocollagen is a matrix that exhibits excellent biocompatibility, and, while an atelocollagen-based delivery system significantly contributes to improving the sustained-release performance and improving the stability within an organism (persistence), there is still room for improving the efficiency of transfection into the cells of an organism. Thus, the atelocollagen-based method of delivering a nucleic acid construct has not necessarily been an entirely satisfactory method for the various modes of gene silencing that use RNA interference. Moreover, it is also desirable that the nucleic acid construct be transfected in a naked state to the maximum extent possible.

To date, the transfection of plasmid DNA into biological tissue by electroporation has been examined and has been successful in the expression of gene products in vivo. With regard, on the other hand, to the delivery into various types of cells of nucleic acid constructs capable of inhibiting gene expression by, for example, RNA interference, e.g., siRNA, it is known that the inhibiting effect on gene expression differs widely depending on the delivery method. In addition, the realization of gene silencing in vivo through the electroporative transfection of a nucleic acid construct that expresses RNA interference, e.g., siRNA, has not been documented.

An object of the present invention, therefore, is to provide an effective technology for transfecting a nucleic acid construct capable of inhibiting gene expression into an organism and to provide uses of this technology. Another object of the present invention is to provide technology for effectively transfecting such a nucleic acid construct by an epidermal route into a target tissue in the neighborhood of the subepidermis and to provide uses of this technology.

The present inventors discovered that gene silencing in an organism can be achieved by the electroporative transfection into the organism of a nucleic acid construct that is capable of inhibiting gene expression. The present invention was achieved based on this discovery. The present invention thus provides the following means.

One aspect of the present invention provides a method of controlling an electroporation apparatus for an animal encompassing humans and nonhuman animals, comprising the step of: applying, in the presence of a nucleic acid construct capable of inhibiting the expression of a gene in the animal, voltage to the electrodes of the aforementioned electroporation apparatus that are disposed at biological tissue of the animal.

The nucleic acid construct in this aspect can be selected from single-stranded and double-stranded DNAs, single-stranded and double-stranded RNAs, DNA-RNA hybrids, and DNA-RNA chimeric oligonucleotides. The nucleic acid construct can also be a nucleic acid construct that is capable of expressing RNA interference in the animal. The nucleic acid construct is preferably siRNA. When the nucleic acid construct is siRNA, the stability in serum is preferably improved by modification, and the nucleic acid construct preferably has a half life in human serum of at least 50 hours. This half life is the half life where the unstabilized nucleic acid construct having the same structure has a half-life in human serum within 2 hours.

The nucleic acid construct in this aspect is preferably a construct that is capable of expressing RNA interference, with a gene that promotes angiogenesis being targeted. A vascular endothelial growth factor gene is a highly suitable example of such a gene.

The biological tissue in this aspect can contain a solid tumor. Moreover, the biological tissue in this aspect can be tissue present in the epidermis or in the subepidermis.

One aspect of the present invention comprises the step of supplying a biodegradable matrix material to the biological tissue or into the vicinity thereof, prior to or after or at the same time as the application of voltage to the electrodes. In addition, the aforementioned voltage application step can be a step in which the voltage is applied to the electrodes disposed against the biological tissue, after or while supplying the nucleic acid construct to the biological tissue or to the vicinity thereof.

The electrodes in this aspect may also comprise at least one plate-shaped electrode and may also comprise one or two or more needle-shaped electrodes. In addition, the needle-shaped electrode may comprise an orifice and a hollow part through which liquid containing the nucleic acid construct can transit. The electroporation apparatus may also comprise a plate-shaped electrode and a needle-shaped electrode as counter electrodes.

The aforementioned plate-shaped electrode may be disposed in the present invention on the surface of the biological tissue into which the nucleic acid construct is to be transfected and the needle-shaped electrode may be disposed puncturing into this biological tissue or into the vicinity thereof. Moreover, the aforementioned biological tissue can be a subcutaneous solid tumor; the plate-shaped electrode can be disposed abutting on the surface of the epidermis that covers the subcutaneous solid tumor; and the needle-shaped electrode can be disposed puncturing into this biological tissue or into the vicinity thereof. The voltage applied to the electrodes can be at least 50 V and no more than 70 V.

According to another aspect of the present invention there is provided a method of controlling an electroporation apparatus for an animal encompassing humans and nonhuman animals, comprising the step of applying, in the presence of siRNA capable of expressing RNA interference in the animal, a prescribed voltage across a needle-shaped electrode that is disposed in the lower portion of subepidermal diseased tissue in the animal and a plate-shaped electrode that is disposed on the surface of the epidermis that covers the diseased tissue. In a preferred aspect thereof, the animal is a human and the diseased tissue contains tissue for which an inhibition of progression, improvement, or treatment is possible through an inhibition of angiogenesis.

According to yet another aspect of the present invention, there is provided a method of producing a nonhuman animal, comprising the step of transfecting, by electroporation, a nucleic acid construct capable of inhibiting the expression of a gene in the nonhuman animal into the cells of biological tissue of the nonhuman animal. In a preferred aspect thereof, the nucleic acid construct is a nucleic acid construct capable of expressing RNA interference, with a disease-associated gene being targeted.

According to another aspect of the present invention there is provided a method of producing a nonhuman animal, comprising the steps of preparing a nonhuman animal that has a pathological condition, genetic mutation, or biological tissue or cell phenotype capable of manifesting as a model of a human disease; and transfecting, by electroporation, a nucleic acid construct capable of inhibiting the expression of a gene in the nonhuman animal into the cells of biological tissue of the nonhuman animal.

According to another aspect of the present invention, there is provided a method of identifying a therapeutic agent, comprising the steps of preparing a nonhuman animal that has a pathological condition, genetic mutation, or biological tissue or cell phenotype capable of manifesting as a model of a human disease; transfecting, by electroporation, a nucleic acid construct capable of inhibiting the expression of a gene in the nonhuman animal into the cells of biological tissue of the nonhuman animal; and analyzing the pathological condition or the aforementioned biological tissue or cell phenotype of the animal model into which the nucleic acid construct has been transfected.

According to another aspect of the present invention, there is provided a method of identifying a therapeutic agent, comprising the steps of transfecting, by electroporation, a nucleic acid construct capable of inhibiting the expression of a gene in a nonhuman animal into the cells of biological tissue of the nonhuman animal to form a pathological condition, biological tissue or cell phenotype for a human disease in at least a portion of the nonhuman animal; and administering one or two or more compounds to the nonhuman animal and analyzing the aforementioned pathological condition or biological tissue or cell phenotype.

According to another aspect of the present invention, there is provided a method of identifying a target compound for drug discovery, comprising the steps of transfecting, by electroporation, a nucleic acid construct that is capable of inhibiting gene expression in a nonhuman animal, with a disease-associated gene in the nonhuman animal being targeted, into the cells of biological tissue of the nonhuman animal; and analyzing the phenotype of the aforementioned cells or biological tissue into which the nucleic acid construct has been transfected.

### BRIEF DESCRIPTION OF THE DRAWINGS

FIG. 1 is a diagram that shows an example of an electroporation apparatus;

FIG. 2 is a diagram that shows process flowchart examples (a), (b), (c), and (d) for the transfection of a nucleic construct into the cells of biological tissue by electroporation;

FIG. 3 is a diagram that shows an example of a process for transfecting a nucleic acid construct using an electroporation apparatus;

FIG. 4 is a diagram of the target site sequences of siRNAs against the mRNA (CDS) of hVEGF-A;

FIG. 5 shows the structures of the individual siRNAs;

FIG. 6 is a diagram that shows the inhibitory activity on hVEGF-A expression in PC-3 cells by the individual siRNAs;

FIG. 7 is a diagram that shows the inhibitory activity on VEGF-A in PC-3 cells by stabilized siRNA, unmodified siRNA, and their scrambled siRNAs;

FIG. 8 is a diagram that shows the anti-tumor effect (therapeutic effect) of siRNA by electroporation;

FIG. 9 is a diagram that shows photographs (a), (b), (c), and (d) of the appearance of tumors on day 40 after the start of treatment;

FIG. 10 is a diagram that shows the image yielded by immunochemical staining using CD31 as a marker of intratumoral microvessel density, for a group receiving stabilized siRNA and a group receiving stabilized, scrambled-sequence siRNA; and

FIG. 11 is a diagram that shows the anti-tumor effect (therapeutic effect) of the siRNA of Example 6 by electroporation.

### BEST MODE FOR CARRYING OUT THE INVENTION

The method of controlling an electroporation apparatus for an animal encompassing nonhuman animals and humans, that is one aspect of the present invention characteristically comprises the step of applying, in the presence of a nucleic acid construct capable of inhibiting the expression of a gene in the animal, voltage to the electrodes of an electroporation apparatus that are disposed against biological tissue of the animal. This method provides a state in which the surfaces of the cells constituting the biological tissue are porated by the application of voltage to electrodes disposed at the biological tissue of the animal, thereby enabling transfection of the nucleic acid construct into the cells. Once it has been transfected into the cells, the nucleic acid construct is then able to inhibit the expression of a prescribed gene.

The method of producing a nonhuman animal that is another aspect of the present invention characteristically comprises the step of transfecting, by electroporation, a nucleic acid construct capable of inhibiting the expression of a gene in the nonhuman animal into the cells of biological tissue of the nonhuman animal. This method, by electroporatively transfecting the nucleic acid construct into the cells of the biological tissue, can produce a nonhuman animal that exhibits inhibited gene expression in those cells.

The method of identifying a therapeutic agent that is yet another aspect of the present invention characteristically comprises the steps of preparing a nonhuman animal that has a pathological condition, genetic mutation, or biological tissue or cell phenotype capable of manifesting as a model of a human disease; transfecting, by electroporation, a nucleic acid construct capable of inhibiting the expression of a gene in the nonhuman animal into the cells of biological tissue of the nonhuman animal; and analyzing the pathological condition or the aforementioned biological tissue or cell phenotype of the aforementioned animal model into which the nucleic acid construct has been transfected. This detection method, through its analysis, for example, of a pathological condition in a nonhuman animal whose cells have been transfected with the aforementioned nucleic acid construct and which therefore exhibits an inhibition of gene expression, enables the facile evaluation of the efficacy of the nucleic acid construct against the aforementioned disease. As a result, this enables screening for nucleic acid constructs that are effective for the treatment or prevention of the disease under consideration.

The method of identifying a therapeutic agent that is still another aspect of the present invention characteristically comprises the steps of transfecting, by electroporation, a nucleic acid construct capable of inhibiting the expression of a gene in a nonhuman animal into the cells of biological tissue of the nonhuman animal to form a biological tissue or cell phenotype or pathological condition for a human disease in at least a portion of the nonhuman animal; and administering one or two or more compounds to the nonhuman animal and analyzing the aforementioned pathological condition or biological tissue or cell phenotype. This detection method, through its analysis, for example, of a pathological condition in the aforementioned nonhuman animal, enables the facile evaluation of the efficacy of the compound(s) against the aforementioned disease. As a result, this enables screening for drugs that are effective for the treatment or prevention of the disease under consideration.

The method of identifying a target compound for drug discovery that is another aspect of the present invention characteristically comprises the steps of transfecting, by electroporation, a nucleic acid construct that is capable of inhibiting gene expression and that targets one or two or more disease-associated genes present in biological tissue or cells of at least a portion of a nonhuman animal, into the aforementioned cells or biological tissue; and analyzing the phenotype of the aforementioned cells or biological tissue into which the nucleic acid construct has been transfected. This detection method, through its analysis of the phenotype of the cells or biological tissue, enables the identification of compounds that, through inhibition of the expression of the aforementioned disease-associated gene(s), undergo an increase in activation or level of expression or undergo a reduction in deactivation or level of expression. Such compounds, or inhibitors of the activity of such compounds, can then be taken up as target compounds for drugs that prevent or treat the disease under examination, and this detection method according to the present invention therefore provides new target compounds for drug discovery and can provide a system for screening for drugs effective for the prevention and/or treatment of the disease under examination.

In all of these aspects of the present invention, the aforementioned nucleic acid construct is electroporatively transfected into cells of biological tissue of an animal. Furthermore, by the use of a needle-shaped electrode as at least one of the electroporation electrodes, the nucleic acid construct can be effectively transfected by an epidermal route into a target tissue in the neighborhood of the subepidermis.

While not intended as a limitation on the present invention, it is presumed that, inter alia, the electroporative transfection efficiency and the size of the nucleic acid construct (Stokes diameter of the molecule) participate in the phenomenon discovered by the present inventors, i.e., the manifestation of an excellent expression-inhibiting effect by the electroporative transfection of, for example, a nucleic acid construct that expresses RNA interference, into the cells of biological tissue. First and foremost, it is thought that electroporation, by enabling transfection of the nucleic acid construct into cells in a very short period of time, can achieve a high intracellular concentration of the nucleic acid construct at the time point of transfection due to the transfection of nucleic acid construct into cells that heretofore would have ended up being degraded before its transfection into the cells, which results in the manifestation of an unexpected expression-inhibiting activity. It is believed that a high expression-inhibiting activity is therefore obtained not only for DNA constructs, but also for relatively low molecular weight nucleic acid constructs, e.g., RNA constructs such as ordinarily unstable siRNA.

In addition, the cell membrane undergoes an electrical poration in the case of electroporation, and it is thought that there is a high affinity by low molecular weight nucleic acid constructs such as siRNA for the porated area yielded by this electrical stimulation or that such nucleic acid constructs pass through easily because they are smaller molecules than, for example, the usual conventional DNA expression vectors. The present inventors have deduced that, when a pore is formed in the cell membrane by electroporation, a relationship with a certain equilibrium state is ordinarily established for this pore diameter and the diameter of the molecule to be introduced. That is, for example, a low molecular weight compound with a molecular weight of about 1000 will easily traverse the pore and enter the cell; however, since this reaction is an equilibrium reaction, there is also the problem that the introduced low molecular weight compound also easily exits from the cell at the same time. A low molecular weight nucleic acid construct, for example, siRNA (molecular weight approximately 13,000), having a favorable molecular weight (molecular diameter), accrues the advantage of being resistant to exiting the cell once it has entered the cell through electroporation. It is thought that this tendency is more significant in the case of electroporation directed against biological tissue than in the case of electroporation at the cell level.

In addition, it is thought that by having a matrix, such as collagen, coexist with the nucleic acid construct, high concentrations of the nucleic acid construct can be maintained in the neighborhood of the cells or tissue to be transfected with the nucleic acid construct.

Best modes for the execution of the present invention are described below for the various aspects of the present invention cited above.

(The Method of Controlling an Electroporation Apparatus)

(The Human and Nonhuman Animals)

The inventive method of controlling an electroporation apparatus relates to the application of an electroporation apparatus with animals encompassing humans and nonhuman animals. For the purposes of this Specification, the nonhuman animals encompass nonhuman primates, mammals other than primates, and animals such as birds, reptiles, amphibians, fish, and so forth. The mammals can be exemplified by domestic animals such as the rat, mouse, rabbit, pig, sheep, cow, horse, goat, and so forth, and by pet animals such as the dog, cat, and so forth. The fish can be exemplified by the zebrafish, medaka, and so forth. In particular, the rat, mouse, rabbit, dog, pig, zebrafish, medaka, and the like, are preferably used for disease models and research purposes. The humans and nonhuman animals cited for the present invention also encompass any state selected from embryonic, fetal, and postnatal, while the use of postnatal individuals is preferred for the inventive control method, method of producing a nonhuman animal, method of identifying a therapeutic agent, method of identifying a target gene for drug development, and so forth.

(The Biological Tissue)

In the control method according to the present invention, voltage is applied to the electrodes of an electroporation apparatus that are disposed at the biological tissue of an animal as described above. The biological tissue of the animal is not particularly limited. Based on the selection of the configuration of the electrodes disposed at the biological tissue as described herebelow and the implementation of such measures as, for example, incision of the biological tissue for electrode placement, it will be possible to target not only the neighborhood of the epidermis of an animal, but also all of the interior biological tissue. When percutaneous voltage application is under consideration, the biological tissue is preferably the epidermis or subepidermis (immediately below the epidermis or in the neighborhood thereof). The voltage can be easily applied to such a location when at least a portion of an electrode has, for example, a needle shape, and can penetrate the epidermis. Various joints, such as the knee, are examples of biological tissue that supports a facile percutaneous transfection process. Joints are also preferred because they are directly under the epidermis and because they protrude from the body. Otherwise, intrabuccal is also preferred, for example, the periodontal tissue. Depending on the electrode configuration, biological tissue that can be transvascularly accessed, for example, via blood vessels, is also preferred. In addition, biological tissue is also preferred that enables electrode disposition by an oral, rectal, vaginal, or abdominal route through an endoscopic procedure.

Tissue that encompasses a solid tumor is also preferred for the biological tissue. This is because a nucleic acid construct can be transfected into the cells of a solid tumor by the electroporation apparatus. In particular, the method under consideration enables the percutaneous treatment of a solid tumor that is directly under the epidermis or in the neighborhood thereof.

(The Electroporation Apparatus)

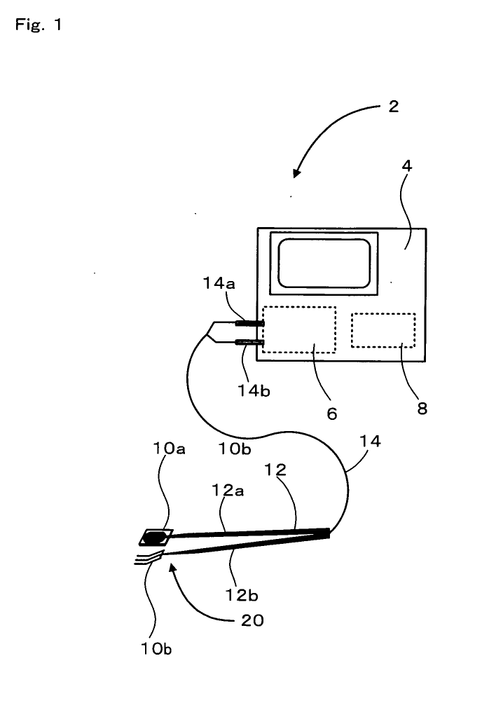

An ordinary electroporation apparatus can be used as the electroporation apparatus in the present invention. An example of an electroporation apparatus 2 is shown in FIG. 1. The electroporation apparatus 2 can comprise a main unit 4 and an electrode member 20 connected to the main unit 4 by a cable 14 and a holder 12. The main unit 4 can be provided with a pulse-generating means 5. The pulse-generating means 6 can apply a voltage to the electrodes 10a, 10b through a conductive wire that runs within and through the cable 10 and the holder 14. Pulse-generating means that can apply an electrical pulse in the form of, for example, an alternating-current (AC) pulse, an exponential pulse, or a direct-current (DC) pulse, using an ordinary alternating-current power source are known for the pulse-generating means 6. While there are no particular limitations here, a pulse-generating means 6 that can apply a DC pulse is preferred. A square wave is preferred for the pulse waveform. A square wave is a wave form that rises sharply to the set voltage, maintains this voltage for a prescribed period of time (the pulse length), and then sharply drops back to a value of 0. The use is more preferred of a pulse-generating means that can apply such a square wave at a plurality of cycles per 1 second, preferably up to 99 cycles per 1 second. Here, one cycle denotes the sum of the pulse on time and the pulse off time.

The main unit 4 can also include an impedance measurement means 8. The impedance measurement means 8 can measure the impedance (resistance value) between the electrodes 10a, 10b prior to or after pulse application.

This apparatus 2 can be provided with at least one pair of electrodes 10a, 10b that are electrically connected to the pulse-generating means 6 of the main unit 4. The electrode 10a shown as an example in FIG. 1 is a rectangular plate-shaped conductor (for example, of platinum), while the electrode 10b has three needle-shaped members arranged in a row to form a fork-shaped configuration as a whole and is disposed so as to face the electrode 10a. Such a plate-shaped electrode is preferably used abutting the biological tissue, e.g., epidermis, mucous membrane, organ, internal body part, and so forth, that is, abutting the biological tissue that is going to be transfected with the nucleic acid construct. With regard to the three needle-shaped members of the electrode 10b, the three needles are gently curved in about the same manner, which facilitates insertion into subcutaneous biological tissue. Needle-shaped electrodes that can be used in the present invention may comprise one or two or more needle-shaped members. The arrangement of a plurality of needle-shaped members is not particularly limited; for example, they can be arranged in a single row in parallel to each other or can be aligned in a plurality of rows. Such a needle-shaped electrode is preferably used inserted into the biological tissue that is going to be transfected with the nucleic acid construct or into the neighborhood thereof.

When a plate-shaped electrode and a needle-shaped electrode are used in the present invention, the plate-shaped electrode can be disposed against the surface of the biological tissue to be transfected with the nucleic acid construct and the needle-shaped electrode can be disposed puncturing into this biological tissue or into the neighborhood thereof. This makes it possible to easily secure and maintain a current level that will facilitate transfection of the nucleic acid construct into the biological tissue and thereby makes it possible to reliably carry out transfection with the nucleic acid construct. Moreover, in instances where the biological tissue is a subcutaneous solid tumor, the plate-shaped electrode can be disposed abutting the surface of the epidermis that covers the subcutaneous solid tumor and the needle-shaped electrode can again be disposed puncturing into this biological tissue or into the neighborhood thereof. This makes it possible to hold the subcutaneous solid tumor stable and as a result makes it possible to reliably and efficiently transfect the nucleic acid construct.

In addition, the nucleic acid construct can be effectively transfected into subepidermal tissue by abutting a plate-shaped electrode against the epidermis and disposing a needle-shaped electrode underneath the subepidermal target tissue, for example, a solid tumor. In particular, in instances where the epidermis has been present in an interposed position, the amount of current has generally been prone to variation and also prone to decline; however, by deploying the electrodes versus subepidermal biological tissue in the manner described above, a sufficient amount of current can be supplied to the construct even at relatively low voltages, thereby securing the transfection efficiency and improving the procedure's low invasiveness.

For the solid tumors generated in the examples, vide infra, the present inventors have found that the use of a plate-shaped electrode in combination with a needle-shaped electrode (disposed so as to sandwich the solid tumor by inserting the needle-shaped electrode in the bottom of the solid tumor and disposing the plate-shaped electrode on the top of the solid tumor) can secure a more favorable amount of current than the passage of current by a plate-shaped electrode combined with another plate-shaped electrode (disposed so as to sandwich the solid tumor). That is, by using a plate-shaped electrode and a needle-shaped electrode (in particular a needle-shaped electrode having at least two needles arranged in a row), or by disposing the electrodes against the surface and in the interior of biological tissue that contains the target cells, the advantage accrues of making possible a stable supply of the intended amount of current to the biological tissue. The stable supply of the intended amount of current is advantageous for effecting a stable and reliable electroporative transfection of the nucleic acid construct.

The electrodes 10a, 10b are not limited to this combination of a plate-shaped electrode with a needle-shaped electrode. As examples of electrode combinations that may be used in the present invention, both may be plate-shaped electrodes or both may be electrodes equipped with one or two or more needle-shaped members. Nor must the electrodes occur as a pair, and one or two or more positive electrodes may be combined with one or two or more negative electrodes. Moreover, the plate portion of the plate-shaped electrode can have various shapes, for example, circular, oval, square, and so forth. When a needle-shaped electrode is employed, a flow path that enables a liquid to pass through can be disposed in its interior and a discharge opening can be disposed at its terminus. In this case, for example, by connecting the end of an injection syringe to the flow path in the needle-shaped electrode and setting up a configuration in which a liquid containing the nucleic construct can be supplied through the flow path, it becomes possible to apply the voltage immediately after or during injection of the nucleic acid construct into the organism. This can prevent diffusion of the nucleic acid construct from the target location.

The electrodes 10a, 10b are preferably connected to the pulse-generating means 6 via a holder 12 and a cable 14. The holder 12 can be grasped and handled with one hand, has a pair of arms 12a, 12b that can be spread apart or closed together, and is provided with an electrode 10a, 10b at the end of each arm. A cable 14 that has a terminal 14a and a terminal 14b at its end may be fixed at the base end of the arm pair 12a, 12b of the holder 12. A conductive wire connected to each of the electrodes 10a, 10b runs within the holder and the cable 14; on the terminal side of the cable 14, the end of each of these conductive wires may form a terminal 14a, 14b and may be electrically connected to the pulse-generating means 6. In this apparatus 2, the electrodes 10a, 10b, the holder 12, and the cable 14 can form an electrode unit (member) 20 that is exchangeable with respect to the main unit 4. As described herebelow, this electroporation apparatus 2 provided with electrodes 10a and 10b can be used as an apparatus for transfecting a nucleic acid construct into the biological tissue (particularly subcutaneous tissue) of a nonhuman animal or a human, and as an apparatus for inhibiting gene expression, and as an apparatus that effects treatment using a nucleic acid construct (particularly an apparatus for treating a subcutaneous disease site, e.g., a subcutaneous solid tumor). In addition, these electrodes 10a and 10b can similarly be used as electrodes for an apparatus for transfecting a nucleic acid construct into the biological tissue (particularly subcutaneous tissue) of a nonhuman animal or a human, for an apparatus for inhibiting gene expression, and for treatment using a nucleic acid construct (particularly electrodes for treating a subcutaneous disease site, e.g., a subcutaneous solid tumor).

(The Nucleic Acid Construct)

The nucleic acid construct in this invention is constructed so as to be capable of inhibiting gene expression. For the purposes of this Specification, inhibition of gene expression denotes an inhibition of gene expression by, for example, an antisense nucleic acid procedure in which transcription is inhibited by interaction, for example, hybridization, with DNA that encodes, for example, a gene; an antisense nucleic acid procedure in which transcription or translation is inhibited by interaction, for example, hybridization, with RNA, for example, mRNA; an RNA interference procedure in which translation is inhibited or a transcript is degraded based on interaction, for example, hybridization, with a transcript, for example, mRNA; a decoy nucleic acid procedure; a ribozyme procedure; and so forth. Moreover, although this is not inhibition of gene expression, aptamers can also be introduced by electroporation. The nucleic acid construct also includes miRNA, e.g., pre-microRNA (miRNA) and mature miRNA, as well as small RNA molecules that control the action of miRNA. In this Specification, “nucleic acid” denotes a polynucleotide such as deoxyribonucleic acid or ribonucleic acid. Moreover, this term encompasses single-stranded (DNA or RNA, sense or antisense) and double-stranded polynucleotides (DNA or RNA). It also encompasses DNA-RNA hybrids (double-stranded) and DNA-RNA chimeric oligonucleotides (single-stranded), peptide nucleic acid (PNA), and morpholinooligonucleotides. The polynucleotide may have also been subjected to natural or artificial modification. For the purposes of this Specification, “RNA interference” denotes a phenomenon in which double-stranded RNA mediates in a sequence-specific manner the degradation of a transcription product, for example, the mRNA from a target gene, or mediates in a sequence-specific manner the inhibition of the translation of, for example, the mRNA from a target gene. As a result, the expression of a target gene can be inhibited by RNA interference. Here, inhibition of the expression of a target gene means the inhibition of the translation of mRNA encoded by the target gene into polypeptide or a reduction in the level of expression of the protein that is the translation product from the mRNA encoded by the target gene.

There are no particular limitations on the target gene. The target gene may be, for example, a gene for the prevention or treatment of a disease or a gene whose functional analysis is required. For example, with the objective of treating, for example, solid tumors, the target gene can be a gene that codes for a substance that induces angiogenesis, most prominently vascular endothelial growth factor (VEGF), which is released by cancer cells and induces angiogenesis, but also fibroblast growth factors such as aFGF and bFGF, tumor necrosis factor cc (TNF-α), angiogenin, and so forth, or a gene that encodes epidermal growth factor (EGF), which promotes the growth of cancer cells. The targeting of these angiogenesis-associated genes makes possible a highly tumor-specific therapy in which side-effects are restrained. Moreover, the prevention or treatment of a variety of diseases caused by angiogenesis is also made possible.

Because, among the preceding, VEGF has a strong angiogenic activity, the VEGF gene is very useful not only as a target gene for cancer treatment, but also as a target gene for the treatment of diseases other than cancer in which a major cause is VEGF overexpression, for example, ocular neovascularizing diseases (e.g., diabetic retinopathy, retinal vein occlusion) and arteriosclerosis. VEGF has a structure in which subunits with a molecular weight of approximately 22,000 form a dimer and promotes the proliferation•migration•lumen formation of vascular endothelial cells and causes the upregulation of angiogenesis•vascular permeability at the organism level. In addition, it induces the upregulated production of coagulation system•fibrinolysis system cofactors, such as tissue factor and plasminogen activator (PA,) and the upregulation of, for example, the expression of matrix metalloproteinase and PA receptors, and also degrades vascular basement membrane (Ferrara, N.: J. Mol. Med, 77, 527-543, 1999). VEGF also upregulates vascular permeability, as shown by its alternate name of VPF. It is known that VEGF (VEGF-A) occurs as 5 isoforms of different size (i.e., VEGF₁₂₁, VEGF₁₄₅, VEGF₁₆₅, VEGF₁₈₉, and VEGF₂₀₆, where the subscripts show the number of structural amino acids) due to alternative splicing (Tischer, E. et al.: J. Biol. Chem., 266: 11947-11954, 1991). Cells that produce VEGF can produce several different subtypes simultaneously. While VEGF₁₂₁ and VEGF₁₆₅ in general predominate, the expression of VEGF₁₈₉ is also seen in many cells. Also useful are nucleic acid constructs, such as siRNA, for which the target gene is one or two or more of the genes for the 5 VEGF subtypes biosynthesized by alternative splicing. VEGF-B and placenta growth factor (PLGF) can also be targeted. Based on the preceding, the nucleic acid construct of the present invention can be targeted to the VEGF family comprising VEGF-A, VEGF-B, PLGF, and so forth. Genes in the VEGFR family, e.g., VEGFR-1, -2, -3, and so forth, can also be targeted. Examples of other cancer-associated genes are the mutated p53 gene and the ezh2 gene.

When the targeted biological tissue is tumor tissue (particularly subcutaneous tumor tissue), and based on the fact that within the tumor angiogenesis is active and vascular permeability is elevated, the use is preferred of a nucleic acid construct for which the target gene is an angiogenesis-associated gene that promotes angiogenesis, for example, for vascular endothelial growth factor (VEGF). However, since the vascular permeability in such tissue is also elevated and much water is present, a nucleic acid construct delivered by electroporation is preferred. In particular, when a plate-shaped electrode is used in combination with a needle-shaped electrode as described below, as a result of the insertion of the needle-shaped electrode into the vicinity of the tumor tissue, the amount of current can be stably and easily secured and maintained due to this vascular permeability and high water milieu, thereby enabling an efficient transfection of the nucleic acid construct.

The target gene need not be a gene endogenous to the human or nonhuman animal that is going to be transfected with the nucleic acid construct. For example, an RNA sequence in an RNA virus can be targeted with the goal of preventing or treating a disease caused by a viral infection. For example, an RNA sequence from the HIV-1 virus, hepatitis C virus, polio virus, Rouse sarcoma virus, papilloma virus, influenza virus, and so forth, can be targeted. A gene that encodes an endogenous factor required for viral proliferation may also be made a target gene in such viral infectious diseases.

Inflammatory disease-associated genes may also be employed as target genes. Examples of inflammatory disease-associated genes are IL-1 (α and β), IL-6, IL-4, and so forth. These are related to inflammatory diseases such as rheumatism and rheumatoid arthritis. Considering that a percutaneous transfection procedure from outside the body is easily effected, targeting a gene related to such inflammatory diseases of the joints is particularly effective for the present invention. Examples of other inflammatory disease-associated genes are the TNFα gene and genes for the TNFα receptor family, which are genes associated with dermatitis, such as atopic dermatitis. The present invention also provides a facile percutaneous transfection procedure for dermatitis diseases.

Anti-apoptosis-associated genes are yet another example of target genes. Anti-apoptosis-associated genes are cancer genes related to the development and progression of cancer. Such genes can be exemplified by the bcl-2 gene family, such as the bcl-2 gene most prominently, but also bcl-x, bcl-w, mcl-1, bfl-1/A1, bax, bad, bik, and so forth. Targeting the bcl-2 gene is particularly preferred. siRNA whose gene target is the bcl-2 gene is described in, for example, Japanese Patent Application Laid-open No. 2005-13199.

The nucleic acid construct capable of expressing RNA interference is constructed to be capable of inhibiting the expression of the target gene by being targeted to at least a portion of the transcription product, e.g., mRNA, from the target gene. One aspect of such a nucleic acid construct is an RNA construct that has a double-stranded structure of hybridized oligoribonucleotide, that is, naked RNA. Specific examples are relatively short double-stranded oligoribonucleotide with or without respective overhanging 3′ ends (small interfering RNA: siRNA) and a single oligoribonucleotide that (has or) forms a hairpin structure (short hairpin RNA: shRNA). These RNA constructs are preferred in that they can directly invoke RNA interference. An RNA construct of single-stranded oligoribonucleotide that does not form a hairpin structure can also express RNA interference.

siRNA has a sense sequence that corresponds to the target sequence and an antisense sequence wherein this sense sequence and antisense sequence are hybridized over a defined length to form a double-stranded structure. That is, the sense sequence and the antisense sequence are each part of a double strand that pairs over a prescribed length. While the sense sequence and the antisense sequence hybridize with each other, a part of each sequence may have a nonpairing region. For example, the sense sequence may have one or several mismatched bases and/or base deletions. The sense sequence and antisense sequence in siRNA may each have an overhanging 3′ end or may lack an overhanging 3′ end (blunt end type). When an overhang is present, the 3′ end overhang of the sense sequence need not agree with the target sequence on the mRNA and the 3′ end overhang of the antisense sequence need not be complementary to the target sequence on the mRNA.

shRNA has on its 5′ side a sense sequence that corresponds to the target sequence, just as for siRNA, while on its 3′ side it has the antisense sequence; these form a stem by pairing over a defined length. shRNA has a loop region between these sequences that has a region that can be processed by nucleases. As a consequence, shRNA undergoes processing within the cell to give an siRNA. Just as described above for siRNA, the sense sequence and antisense sequence corresponding to the overhangs of the siRNA derived from shRNA also need not agree or be complementary, respectively, with the target sequence.

The length in the nucleic acid construct of this aspect of the double strand yielded by the pairing of the sense sequence and antisense sequence is not particularly limited as long as an RNA interference activity is obtained; however, it is preferably no greater than 50 base pairs and typically is preferably 13 to 28 base pairs and more preferably is 13 to 27 base pairs and even more preferably is 19 to 21 base pairs. 19 or 20 base pairs is most preferred. A sense sequence that has a 3′-side structure that does not form a double strand and an antisense sequence that has a 3′-side structure that does not form a double strand, typically preferably have 15 to 30 nucleotides, more preferably 15 to 29 nucleotides, and even more preferably 21 to 23 nucleotides. 21 or 22 nucleotides is most preferred. The 3′ end overhang in siRNA is preferably 2 to 4 nucleotides and more preferably is 2 nucleotides. Moreover, the loop region in shRNA preferably has a length sufficient to avoid impeding the formation and maintenance of the double strand (the stem of shRNA) and the 3′ end structure in this aspect.

The target sequence of the target gene for the nucleic acid construct of this aspect can be determined, for example, by the appropriate application of the following rules and a suitable siRNA or shRNA can thereby be designed.

(1) Target a sequence in the CDS of the target gene that has, for example, AA(N19)TT, AA(N21), or NA(N21) (use the 19 bases from the 3rd base to the 21st base in this sequence as the siRNA);

(2) in order to avoid transcription factor binding regions, use the downstream side that is at least 50 to 100 bases downstream from the start codon;

(3) check the predicted secondary structure of the target mRNA in order to avoid regions that would be difficult to bind due to steric hindrance;

(4) based on an homology search in BLAST and so forth, select sequences that do not exhibit homology with other genes; and

(5) use a GC content of around 50% (in particular, 47 to 52%).

In addition to the preceding, the procedure for designing siRNA, including the procedure for determining the target sequence, can be carried out through the appropriate application of the various rules disclosed, for example, at http://design.rnai.jp/sidirect/index.php, http://www.rockefeller.edu/labheads/tuschl/sirna.html, “Rational siRNA design for RNA interference” (Nature Biotechnology, vol. 22, 326-330 (2004), Angela Reynolds, Devin Leake, Queta Boese, Stephen Scaringe, William S. Marshall, & Anastasia Khvorova), and “Improved and automated prediction of effective siRNA” (Biochem. Biophys. Res. Commun., 2004 Jun. 18; 319(1):264-74, Chalk A M, Wahlestedt C, Sonnhammer E L).

For example, the VEGF siRNAs #1 to #4 shown in Japanese Patent Application Laid-open No. 2004-313141 are examples of siRNA that can inhibit the expression of human VEGF mRNA (GenBANK accession number NM_{—}003376, Leung, D. W. et al.: Science, 246, 1306-1309, 1989; Keck, P. J. et al.: Science, 246, 1309-1312). Each of these siRNAs has a 2 nucleotides overhang region at the 3′ end on both the sense sequence and the antisense sequence. The target sequences for these siRNAs are target sequences obtained by selecting fourteen 21-base candidate sequences with a GC content of 45 to 55% from target sequences having AA or CA at the 5′ terminal; narrowing the candidates down to 7 sequences that exhibited relatively little “GC deviation”; and for these 7, carrying out a BLAST search for similar sequences and selecting targets with little sequence similarity; those for which relatively little effect from steric hindrance was predicted considering the results of an analysis of the secondary structure in the neighborhood were used as the target sequences.

The nucleic acid construct under consideration can be synthesized by known methods for the chemical synthesis of polyribonucleotides, for example, the phosphoamidite method. It can also be synthesized using an in vitro transcription technique. For example, DNA encoding the pertinent polyribonucleotide can be synthesized; utilizing PCR on this DNA and using a primer that has an RNA promoter sequence, such as the T7RNA promoter, and using DNA polymerase, a double-stranded DNA template for transcription can then be synthesized; and, using RNA polymerase, an in vitro transcription reaction can be carried out on this double-stranded DNA transcription template. This will yield the desired single-stranded RNA. In the case of siRNA, the sense RNA and antisense RNA obtained in this manner can be hybridized to produce double-stranded RNA; appropriate terminal degradation can be carried out, for example, with RNase; and the resulting double-stranded RNA can be purified to yield the siRNA. In the case of shRNA, a single-stranded RNA can be produced that has a sense sequence, antisense sequence, and, between these two sequences, a loop sequence that can be trimmed by various base-specific RNases, and the shRNA can be obtained by annealing the sense region with the antisense region. siRNA can also be obtained by treating the loop sequence of the obtained shRNA with a base-specific nuclease. The in vitro transcription method is not limited to this and a variety of methods are known; moreover, the in vitro transcription method can be carried out using various commercially available in vitro transcription kits.

An RNA construct that has a half-life in human serum of at least 30 hours, preferably at least 50 hours, more preferably at least 60 hours, and even more preferably at least 80 hours is used as the RNA construct, e.g., siRNA or shRNA. The use of such a nucleic acid construct makes possible a facile continuation of the RNA interference activity and enables a reduction in the number of administrations. This half-life is the half-life where the half-life of the identical unstabilized, unmodified RNA construct in the same human serum is within 2 hours. A thusly stabilized RNA construct of this type can also be provided with various known modifying groups for nuclease resistance, which may be provided in any part of the polynucleotide. This modification can, for example, bring about stabilization against degradation of the 3′ overhang regions. For example, these can be selected so as to comprise purine nucleotides and particularly adenosine or guanosine nucleotide. Moreover, the nuclease resistance of the overhang in tissue culture can be significantly strengthened by the absence of the 2′ hydroxyl group. The nucleic acid construct of this type can contain at least one modified nucleotide. The modified nucleotide can be disposed in a position at which the target specific activity, for example, the RNA interference-mediated activity, is not substantially affected, for example, within the 5′ end region or the 3′ end region of a double-stranded RNA molecule. In particular, the overhang can be stabilized by the incorporation of a modified nucleotide analog. Preferred nucleotide analogs are selected from sugar-modified ribonucleotides and backbone chain-modified ribonucleotides. However, ribonucleotides in which the nucleic acid base has been modified, that is, ribonucleotides that contain a non-naturally occurring nucleic acid base, infra, rather than a naturally occurring nucleic acid base, are also suitable. The non-naturally occurring nucleic acid base can be exemplified by uridine and thymidine modified at the 5 position, for example, 5-(2-amino)propyluridine and 5-bromouridine; adenosine and guanosine modified at the 8 position, for example, 8-bromoguanosine; deazanucleotides, for example, 7-deazaadenosine; and O- and N-alkylated nucleotides, for example, N⁶-methyladenosine. Preferred sugar-modified ribonucleotides have the 2′ OH group substituted by a group selected from the group consisting of H, OR, halogen, SH, SR, NH₂, NHR, NR₂, and CN, wherein this R is C₁ to C₆ alkyl, alkenyl, or alkynyl, and the halogen is F, Cl, Br, or I. In a preferred backbone chain-modified ribonucleotide, the phosphodiester group that bonds to the adjacent ribonucleotide is replaced with a modifying group, for example, a phosphorothioate group.

Another aspect of the nucleic acid construct is a vector that expressible encodes an RNA construct of the preceding aspect, that is, siRNA or shRNA. The nucleic acid construct of this aspect is preferred in that it enables the continuous expression of RNA interference. With regard to an shRNA-expression vector according to this aspect, the antisense sequence, sense sequence, and also the loop sequence can be constructed in such a manner that a continuous single-stranded RNA that can build the shRNA, is transcribed by intracellular transcription. With regard to an siRNA-expression vector, this can be constructed in such a manner that RNA having a prescribed sense sequence and RNA having a prescribed antisense sequence are transcribed. In the case of an siRNA-expression vector, both the sense sequence and antisense sequence can be expressed by one and the same vector or the sense sequence and antisense sequence can be expressed by different vectors.

The promoter used in such an expression vector may be a polII system or polIII system when a promoter is sought that can produce RNA corresponding to the particular DNA described above. The polIII system promoters can be exemplified by the U6 promoter, tRNA promoter, retroviral LTR promoter, adenovirus va1 promoter, 5SrRNA promoter, 7SK RNA promoter, 7SL RNA promoter, H1 RNA promoter, and so forth. The polII system promoters can be exemplified by the cytomegalovirus promoter, T7 promoter, T3 promoter, SP6 promoter, RSV promoter, EF-1α promoter, β-actin promoter, γ-globulin promoter, SRα promoter, and so forth.

The expression vector can have the form of a plasmid vector or a viral vector. There are no particular distinctions with regard to the type of vector, which can be selected in conformity to, for example, the cell to be transfected. For example, in the case of mammal cells, examples are viral vectors such as retrovirus vectors, adenovirus vectors, adeno-associated virus (AAV) vectors, vaccinia virus vectors, lentivirus vectors, herpes virus vectors, alphavirus vectors, EB virus vectors, papilloma virus vectors, foamy virus vectors, and so forth.

A nucleic acid construct that adopts this expression vector aspect can be easily constructed based on commercially available vectors that have been constructed to support siRNA or shRNA production, or based on the protocols for such vectors, or based on Revised RNAi Experimental Protocols (supplement to Experimental Medicine, published 1 Oct. 2004, Yodosha Co., Ltd.), and so forth.

The nucleic acid construct may be an antigene nucleic acid, an antisense nucleic acid, a decoy nucleic acid, or a ribozyme. Antigene nucleic acid is DNA or RNA that has a base sequence complementary to a DNA and that, by forming a double strand or triple strand with the DNA, inhibits expression of the gene encoded by the DNA. Antisense nucleic acid has a base sequence complementary to an RNA (genomic RNA or mRNA) and, by forming a double strand therewith, inhibits the expression (transcription, translation) of the genetic information encoded by the RNA. The antisense sequence need not be entirely complementary to the target sequence as long as it can block translation or transcription of the gene; moreover, the antisense sequence may employ, for example, modified bases. As a general matter, the length of the antisense nucleic acid sequence being designed is not particularly limited as long as gene expression can be inhibited, and may be exemplified by 10 to 50 bases and preferably 15 to 25 bases. Decoy nucleic acid (RNA) is RNA that has the sequence of a gene that codes for protein that binds a transcription factor, or that has the sequence of the binding site for a transcription factor, or that has a sequence similar to the preceding sequences; these inhibit the action of a transcription factor through their introduction into the cell as “decoys”. A ribozyme cleaves the mRNA for a specific protein and thereby prevents the translation of this protein. Ribozymes can be engineered based on the gene sequence that encodes the specific protein; for example, the method described in FEBS Letters, 228; 228-230 (1988) can be used for hammerhead ribozymes. Not only hammerhead ribozymes, but also ribozymes that cleave the mRNA of a specific protein, for example, hairpin ribozymes, delta ribozymes, and so forth, can be used in the present invention as long as the ribozyme can inhibit the expression of the specific protein.

(Transfection of the Nucleic Acid Construct)

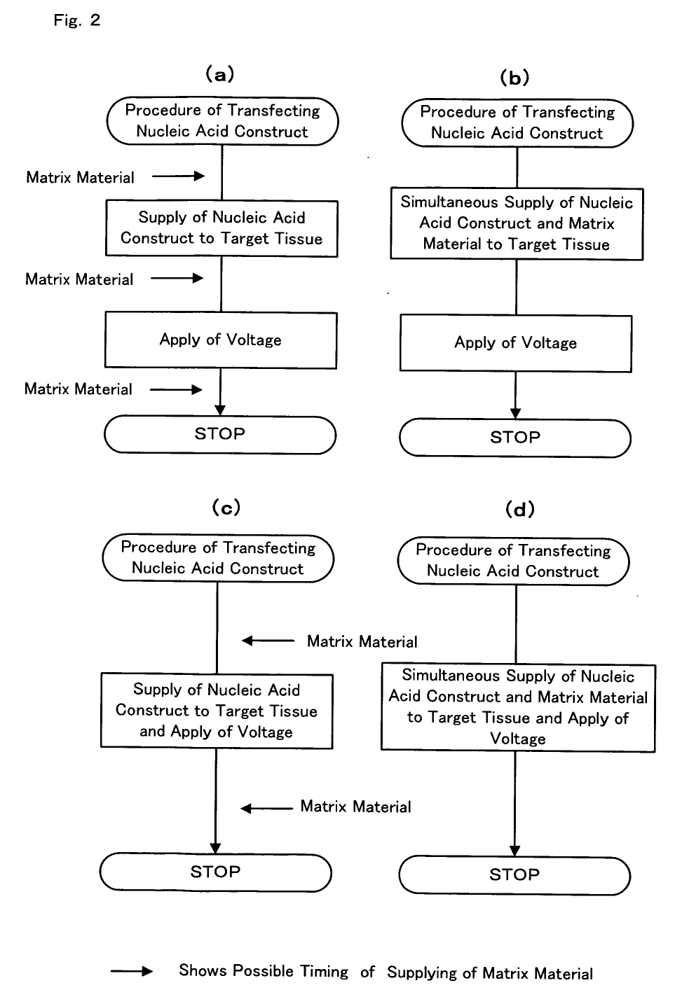

The procedure of transfecting a nucleic acid construct as described above into the cells within an organism using an electroporation apparatus will now be considered, as will control of the electroporation apparatus. Flowcharts of examples of this procedure are shown in FIG. 2. Four types of flowcharts are shown in FIG. 2; these differ in the timing of voltage application and the mode of matrix material addition. In the flowcharts in FIGS. 2(a) and (b), the voltage is applied after the nucleic acid construct has been supplied to and reached the target tissue, while in the flowcharts in FIGS. 2(c) and (d), the voltage is applied accompanying the nucleic acid construct's supply to and arrival at the target biological tissue. These process flowcharts have been laid out so as to facilitate the description, and combinations of the procedures shown in these 4 flowcharts may be used. In addition, combinations of parts of these 4 flowcharts may be employed.

The nucleic construct is supplied to the biological tissue prior to application of the voltage or substantially at the same time as application of the voltage. The nucleic acid construct may be supplied to the target biological tissue by any mode. For example, injection, infusion, nasopharyngeal inhalation, percutaneous absorption, per os, and so forth can be used. The nucleic acid construct may be supplied systemically or locally. When the nucleic acid construct is administered, for example, by venous injection or per os, the use is preferred of a system that effects delivery to the target biological tissue. In addition, a surgical procedure accompanied by, for example, incision of the epidermis, or a percutaneous transluminal procedure may be used to supply the nucleic acid construct. An endoscopic procedure can also be used.

The nucleic acid construct may be supplied to the periphery of the target biological tissue or may be supplied to the interior of the target biological tissue. In addition, the nucleic acid construct may be supplied to a location such that, considering the direction of voltage application, relatively more of the nucleic acid construct moves in the direction of the biological tissue. The specific supply site for the nucleic acid construct is established as appropriate in relation to the electrode configuration and the target tissue.

The nucleic acid construct is preferably supplied to the biological tissue accompanied by a suitable medium. For example, in the case of injection and infusion, a biocompatible medium can be used, such as physiological saline solution or a prescribed buffer. In the case of a local injection, for example, a vasoconstrictor can be supplied, either separately or in the medium, in order to inhibit diffusion of the nucleic acid construct from the biological tissue prior to voltage application. In the case of per os, nasopharyngeal inhalation, percutaneous absorption, and the like, a medium can be used in conformity with the type of formulation for these forms of administration.

The nucleic acid construct may also be supplied together with a matrix material that can form a biodegradable matrix in the biological tissue. The presence of such a matrix material in the biological tissue can inhibit degradation of the nucleic acid construct, inhibit diffusion of the nucleic acid construct from the site of supply, and raise the electroporative transfection efficiency. Examples of matrix material that can provide a biodegradable matrix at the biological tissue are biopolymeric materials such as polysaccharides, e.g., starch, alginic acid, hyaluronic acid, chitin, chitosan, pectinic acid, agarose, derivatives of the preceding, and so forth, and various types of collagen (there are no limitations on the type of collagen and its extraction procedure), e.g., gelatin, atelocollagen, and so forth. Moreover, polymers such as thermosensitive polymers, polylactic acid polymers (e.g., polylactic acid/glycolic acid), and so forth, can be used, as can, for example, Matrigel™ (BD Japan Co., Ltd.) and Pluronic™ (BASF Japan).

Collagen can preferably be used as this matrix material. Soluble collagen and solubilized collagen can be used as the collagen. “Soluble collagen” refers, inter alia, to collagen that is soluble in acidic or neutral water or in water that contains a base. “Solubilized collagen” refers to, for example, enzymatically solubilized collagen that has been solubilized by the action of an enzyme and base-solubilized collagen that has been solubilized by the action of a base.

There are no particular distinctions with regard to the source of the collagen, and collagen extracted from vertebrates, or a recombinant product thereof, can be used. For example, collagen extracted from mammals, fowl, or fish, or a recombinant product thereof, can be used. Collagen includes types I, II, III, IV, and V, and these can be used without particular limitation. An example thereamong is the collagen I yielded by acid extraction from the dermis of mammals, or a recombinant product thereof. More desirable examples are the collagen I obtained by acid extraction from calf dermis and collagen I produced by genetic engineering. Collagen I produced by genetic engineering preferably originates from calf dermis or human dermis. Atelocollagen, which for reasons of safety has had the highly antigenic telopeptide enzymatically removed, is also preferred, as is atelocollagen produced by genetic engineering, while atelocollagen having no more than 3 tyrosine residues per molecule is even more preferred.

When collagen is used as the matrix material, it can be used, for example, at approximately from 0.001 v/v % to no more than 10 v/v %, preferably at approximately from 0.1 v/v % to no more than 5 v/v %, and more preferably at approximately from 1.5 v/v % to no more than 3.75 v/v %.

The biodegradable matrix material under consideration may, as shown by the flowcharts in FIGS. 2(b) and (d), be supplied to the biological tissue at substantially the same time as the nucleic acid construct, or may, as shown by the flowcharts in FIGS. 2(a) and (c), be supplied to a biological tissue separately from the nucleic acid construct and prior to the supply of the nucleic acid construct to the tissue and its arrival thereat. Furthermore, as shown by the flowcharts in FIGS. 2(a) and (c), the matrix material may be supplied to biological tissue after the nucleic acid construct has been supplied to or has reached that site. In those instances where the presence of the matrix material might lower the transfection efficiency of the nucleic acid construct, the matrix material may be supplied to the biological tissue after application of the voltage. The matrix material is preferably locally administered to the target biological tissue by, for example, injection, infusion, and so forth.

In this method, the voltage is applied in the presence of the nucleic acid construct to electrodes disposed at the biological tissue. This results in poration of the cell membrane and transfection of the nucleic acid construct into the cells. There are two timing modes for voltage application. In one timing mode, the nucleic acid construct is supplied to and reaches the biological tissue in advance prior to voltage application and voltage is applied under prescribed conditions to the electrodes disposed in a prescribed location at the biological tissue, at the same time as or after (preferably soon after) the supply of the nucleic acid construct to and its arrival at the biological tissue (FIGS. 2(a) and (b)). In the other timing mode, the voltage is applied while the nucleic acid construct is being supplied to the biological tissue (FIGS. 2(c) and (d)). In the case of voltage application by the latter timing mode, the electrodes are placed in advance in a prescribed location at the biological tissue, and, while in this state, the voltage is applied timed to be during the supply of the nucleic acid construct to and its arrival at the biological tissue. For either mode, placement of the electrodes at the biological tissue is preferably carried out on an appropriate schedule prior to voltage application.

The voltage applied to the electrodes can be at least 50 V but not more than 70 V. A current of about 0.2 A can be secured and maintained in the target biological tissue, e.g., a solid tumor, in this range. The present inventors have found that such a current level is well suited for the transfection of nucleic acid constructs such as, for example, siRNAs. It is difficult to obtain transfection of the nucleic acid construct at below 50 V, while exceeding 70 V is strongly cytotoxic and destroys the tissue.

The electrodes can be disposed in various configurations with respect to the biological tissue, depending on, for example, the location of the biological tissue, the shape of the electrodes, and so forth. When, for example, the biological tissue contains diseased tissue, for example, a solid tumor, the electrodes may be disposed in such a manner that this diseased tissue is sandwiched by at least two electrodes, wherein at least one needle-shaped electrode punctures into the diseased tissue region while the remaining electrode or electrodes abut the periphery of the diseased tissue. In those instances where the electrodes are disposed at diseased tissue, such as a subepidermal solid tumor, a needle-shaped electrode may penetrate to the underside of the bottom of the diseased tissue while another electrode abuts the epidermis that covers the diseased tissue. For biological tissues such as blood vessels, the electrodes are disposed in such a manner that the blood vessel is sandwiched by a pair of plate-shaped electrodes. As necessary, these various electrode disposition configurations can be appropriately established by the individual skilled in the art.

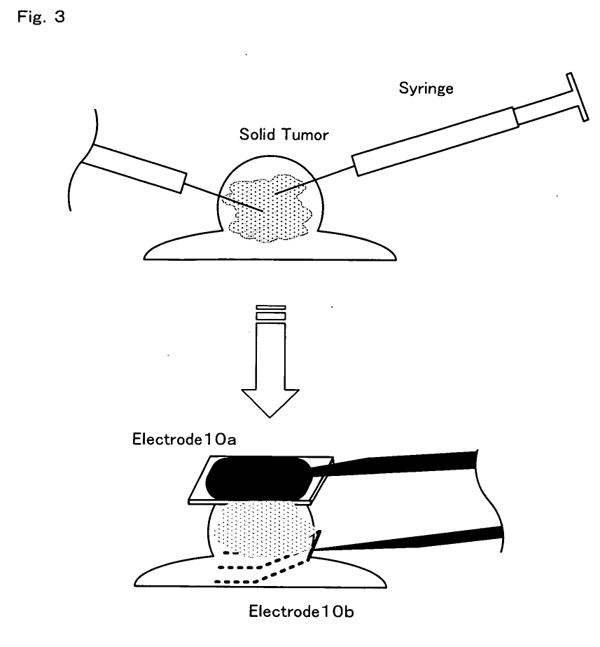

An example is shown in FIG. 3 of a process for applying voltage in the presence of a nucleic acid construct, e.g., siRNA, where the target biological tissue is a solid tumor directly under the epidermis; this example is based on the flowcharts on the left in FIG. 2 and uses the electroporation apparatus 2 shown in FIG. 1. A liquid containing the nucleic acid construct is first injected across the epidermis into the central region of the solid tumor. The injection needle is inserted, and the aforementioned liquid is injected, into at least two locations in the central region of the solid tumor, so as to provide a uniform supply and delivery of the nucleic acid construct into the solid tumor.

Then, a needle-shaped electrode 10b is inserted to the underside of the bottom of the solid tumor into which the nucleic acid construct has been supplied; a plate-shaped electrode 10a is abutted on the epidermis covering the top of the solid tumor; the arms 12a, 12b of the holder 12 are forcibly grasped in such a manner that the electrodes 10a, 10b stably maintain a constant gap to the maximum extent possible; and while in this configuration a square wave pulse is applied for a prescribed period of time at a prescribed cycle by the operation of the pulse-generating means 6 of the main unit 4. This results in poration of the cells within the biological tissue to which the voltage is applied and transfection of the cells by the nucleic acid construct through these openings. By applying the voltage with the electrode 10b connected to the negative terminal of the pulse-generating means 6 and the electrode 10a connected to the positive terminal of the pulse-generating means 6, it is thought that the negatively charged nucleic acid construct supplied around the bottom of the solid tumor is transfected into the interior of the cells through the pores in combination with its displacement to the top of the solid tumor.

In accordance with this disposition of the plate-shaped electrode 10a and needle-shaped electrode (preferably fork-shaped) 10b, it is presumed that, due to the penetration by the needle-shaped electrode 10b into the tissue, the electrical resistance is lower than for the use of a pair of plate-shaped electrodes. In addition, by having the needle-shaped electrode 10b and the plate-shaped electrode 10a face each other, the biological tissue, e.g., a solid tumor, can be securely sandwiched and also sandwiched so as to provide a constant inter-electrode distance, regardless of whether the subcutaneous tissue is directly sandwiched by the two electrodes or is sandwiched with the needle-shaped electrode 10b disposed subcutaneously and the plate-shaped electrode 10a disposed abutting the epidermis. This is presumed to enable a high and stable current level to be readily obtained at a relatively low voltage. Furthermore, since angiogenesis is active within the tumor and the vascular permeability is also high, it is thought that the current level can be readily secured when the needle-shaped electrode 10b is inserted into the vicinity of such tissue. Based on the preceding, it can be concluded that electroporation is preferably executed using the combination of a plate-shaped electrode 10a and a needle-shaped electrode 10b to directly sandwich the biological tissue or to indirectly sandwich subcutaneous tissue across the epidermis in a low-invasive technique.

By employing the method of the present invention to control the electroporation apparatus in the process provided above as an example, one or two or more species of nucleic acid constructs capable of inhibiting gene expression, for example, by RNA interference, can be easily and rapidly delivered in large amounts into the cells of a target biological tissue. Furthermore, a naked nucleic acid construct can be delivered without necessarily also requiring a vehicle such as a viral vector or atelocollagen. This makes it possible to effectively inhibit expression of a target gene through the expression of, for example, an RNA interference activity. Moreover, the transfection of a plurality of nucleic acid constructs is also made easy. Furthermore, by having a matrix, such as collagen, be present with the nucleic acid construct, the stability of the nucleic acid construct itself is improved and, in addition, the transfection efficiency can be maintained and improved by, inter alia, making it possible for the nucleic acid construct to be retained in the vicinity of the target tissue during voltage application.

This method, because it can inhibit the expression of a target gene using a nucleic acid construct, is also able to provide a method of preventing or treating a disease by inhibiting the expression of a target gene and a method for ameliorating morbidity by inhibiting the expression of a target gene. When, in particular, a nucleic acid construct is used that targets a gene the promotes angiogenesis, angiogenesis can be inhibited in a biological tissue, for example, tumor tissue, into which the nucleic acid construct has been transfected. When angiogenesis can as a result be inhibited by inhibiting the expression of the target gene, there are also provided a method of inhibiting the progression of, preventing, or treating a cancer, for example, a solid tumor; a method of inhibiting the progression of, preventing, or treating, for example, a disease caused by angiogenesis, such as an ocular neovascularizing disease (e.g., diabetic retinopathy, retinal vein occlusion) and arteriosclerosis; and a method of inhibiting the progression of, preventing, or treating inflammatory diseases, e.g., atopic dermatitis and rheumatism. In instances where the target gene is a viral gene or an endogenous gene associated with viral proliferation, a method of inhibiting the progression of, preventing, or treating virally induced infectious diseases is also provided.

(The Method of Producing a Nonhuman Animal)

The method according to the present invention of producing a nonhuman animal is characterized by electroporatively transfecting, into the cells of biological tissue of a nonhuman animal, one or two or more species of nucleic acid constructs capable of inhibiting gene expression in the nonhuman animal by, for example, RNA interference. That is, this production method is an aspect in which the inventive method of controlling an electroporation apparatus is applied only to nonhuman animals. Accordingly, this method of producing a nonhuman animal also employs, for its nonhuman animals, nucleic acid constructs, electroporation apparatus and operation thereof, and so forth, all of the scope described hereinabove.

Using this method of producing a nonhuman animal, a nonhuman animal can be obtained, for example, in which the expression of a desired gene is inhibited in a location-specific manner. Even with regard to the inhibition of the expression of a gene where embryonic manipulation would be lethal, such an inhibition of gene expression can be easily achieved, for which reason widespread application is anticipated. Moreover, since 2 or more genes can be easily knocked down by this method, research applications based on inhibiting the expression of two or more genes exist for complex diseases.

When a nucleic acid construct is used that is targeted to a gene that promotes angiogenesis, a nonhuman animal can be obtained in which angiogenesis is inhibited in biological tissue thereof. Such a nonhuman animal enables facile evaluation of, for example, the therapeutic efficacy due to an inhibition of angiogenesis, the efficacy of a combination with another therapy, and so forth. The nonhuman animal in the method of the present invention of producing a nonhuman animal is in particular preferably a post-partum individual. The nonhuman animal under consideration has, for example, biological tissue or cells in which the expression of one or two or more target genes is inhibited.

The method of the present invention of producing a nonhuman animal as described above can easily knock down one or two or more genes in desired cells or in a desired biological tissue. In addition, when a nucleic acid construct, for example, siRNA, is used that is targeted to a disease-associated gene and that has the capacity to inhibit gene expression by, for example, RNA interference, the obtained nonhuman animal will become a transient or local animal disease model, and because of this, a screening system for detecting therapeutic agents using such a nonhuman animal is also provided.

To produce the nonhuman animal, a nonhuman animal that has a pathological condition, genetic mutation, or biological tissue or cell phenotype that provides a model of a human disease, for example, an animal disease model, can be prepared as the nonhuman animal, and this nonhuman animal can be transfected with one or two or more nucleic acid constructs capable of inhibiting gene expression by, for example, RNA interference, and targeted to a gene or genes associated with the disease. The efficacy of the nucleic acid construct(s) can then be evaluated by analyzing the biological tissue or cell phenotype or the pathological condition of the nonhuman animal obtained by the instant method of producing a nonhuman animal. The nonhuman animal used for transfection of the nucleic acid construct may be a commercially available animal disease model, or a normal nonhuman animal may be subjected to a pretreatment that will invoke a model of a human disease in the nonhuman animal. For example, a tumor can be artificially induced to form in a nonhuman animal or a nonhuman animal can be infected with a virus. A tumor can be artificially formed by, for example, administering a mutagen, infecting with a virus, or transplanting tumor cells or tumor tissue; the tumor cells then proliferate at the transplant site or the tumor tissue grows at the transplant site or a metastatic region is formed that has the transplant site as the primary focus.

In this Specification, analysis of the pathological condition and analysis of the biological tissue or cell phenotype encompass all analyses for the purpose of evaluating whether the disease has been ameliorated, treated, or prevented. For example, with regard to an analysis of the pathological condition, the food intake, excretion, activity, and so forth can be monitored; the appearance of the organism as a whole or the appearance of a portion of the body can be monitored; and so forth. With regard to an analysis of the biological tissue or cell phenotype, the biological tissue or cells can be monitored; the expression or activation of a tissue-specific or cell-specific gene or protein can be measured; a metabolite can be measured; and so forth.

(The Method of Identifying a Therapeutic Agent: Gene Therapy Agents)

This method according to the present invention of identifying a therapeutic agent is characterized by electroporatively transfecting, into the cells of biological tissue of a nonhuman animal that has a pathological condition, genetic mutation, or biological tissue or cell phenotype that provides a model of a human disease, one or two or more species of nucleic acid constructs capable of inhibiting gene expression by, for example, RNA interference, in the nonhuman animal; and analyzing the pathological condition or biological tissue or cell phenotype of the aforementioned nonhuman animal into which the nucleic acid construct has been transfected. Due to its use of electroporative transfection of the nucleic acid construct, this methods enables facile evaluation of the efficacy of the transfected nucleic acid construct for the aforementioned disease. As a result, the present invention provides an effective screening system capable of selecting nucleic acid constructs for use as gene therapy agents.

This identification method according to the present invention employs, for its nonhuman animals, nucleic acid constructs, electroporation apparatus and operation thereof, and so forth, all of the scope described hereinabove. Moreover, the nonhuman animal having a genetic mutation or pathological condition that provides a human disease model can be produced by the procedures described above in relation to the method of producing a nonhuman animal.

(The Method of Identifying a Therapeutic Agent: Drugs Such as Low Molecular Weight Compounds)

This method according to the present invention of identifying a therapeutic agent is characterized by electroporatively transfecting, into the cells of biological tissue of a nonhuman animal, one or two or more species of nucleic acid constructs capable of inhibiting gene expression in the nonhuman animal by, for example, RNA interference, to form a biological tissue or cell phenotype or pathological condition for a human disease in at least a portion of the nonhuman animal; and administering one or two or more compounds to the nonhuman animal and analyzing the aforementioned pathological condition or biological tissue or cell phenotype. This method, because it proceeds by forming a human disease condition in a nonhuman animal by electroporative transfection of the nonhuman animal with a nucleic acid construct, administering a compound, and analyzing the result, enables the facile evaluation of the efficacy of, for example, low molecular weight compounds, against the disease condition formed in the nonhuman animal by the nucleic acid construct. As a result, an effective screening system can be provided that can select drugs whose effective component is a low molecular weight compound. The mode of administration of the drug candidate, for example, a low molecular weight compound, is not particularly limited, and the various heretofore known modes can be used, for example, per os, injection, infusion, and so forth.

(The Method of Identifying a Target Compound for Drug Discovery)

The method according to the present invention of identifying a target compound for drug discovery is characterized by electroporatively transfecting, into the cells of biological tissue of a nonhuman animal, one or two or more species of nucleic acid constructs targeted on a disease-associated gene or genes in the nonhuman animal and capable of inhibiting gene expression by, for example, RNA interference; and analyzing the phenotype of the aforementioned cells or biological tissue into which nucleic acid construct has been transfected. This method, through its analysis of the phenotype of the biological tissue or cells, can identify target compounds for the prevention or treatment of the disease under consideration. Based on this target compound identification, a screening system is provided that identifies, for example, compounds that activate or inhibit the aforementioned compound. The analysis of biological tissue or cell phenotype in this case can be exemplified by observation of the cells or biological tissue, measurement of the state of cellular activity, measurement of the level of protein expression, measurement of protein activation, measurement of various metabolites, and so forth. Here, the disease-associated gene may not only be a gene that is clearly related to a disease, but also a gene that is potentially associated with the disease.

As described in the preceding, each of the different embodiments of the present invention—by electroporatively transfecting at least a portion of the cells or tissue of an animal with one or two or more species of nucleic acid constructs capable of inhibiting gene expression by, for example, RNA interference—can simply and rapidly effect knockdown in the organism by gene silencing based on, for example, RNA interference. In particular, direct gene silencing in an organism is made possible by electroporating an RNA construct of siRNA or shRNA and more preferably siRNA directly into the organism.

### EXAMPLES

The present invention is specifically described below through examples, but the examples provided below do not limit the present invention.

### Example 1

Five siRNAs were produced in this example based on the CDS sequence of human VEGF-A (referred to below as hVEGF-A, GenBank Accession Number NM_{—}003376) and their in vitro knockdown activities were investigated.

<siRNA Preparation>

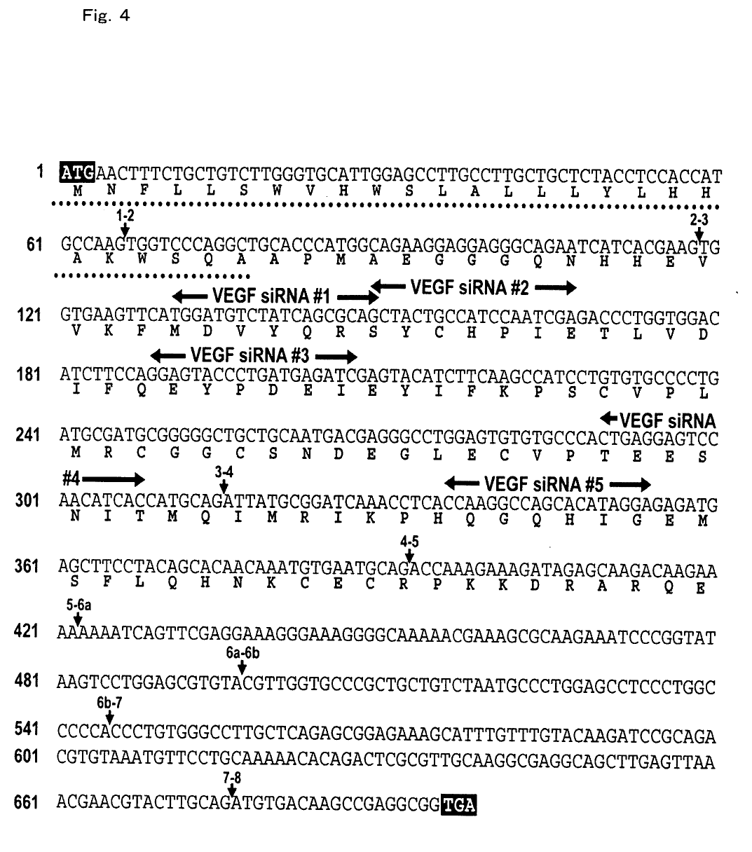

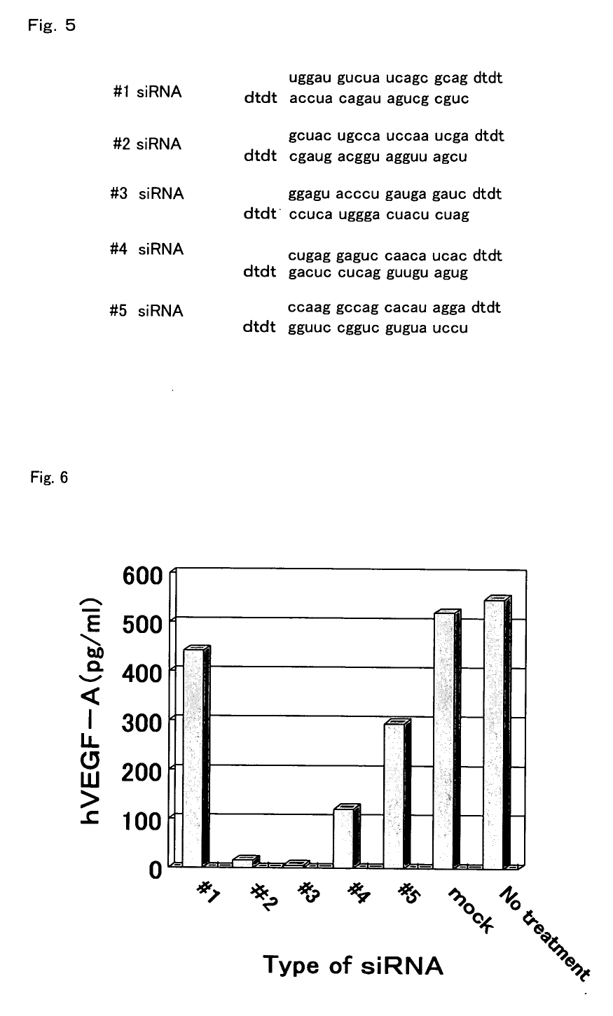

The hVEGF-A target sequences for the 5 siRNAs are shown in FIG. 4 and SEQ ID NO:1 to SEQ ID NO:5. The gene sequence in FIG. 4 is the CDS. The sense sequences of the synthesized siRNAs are shown in SEQ ID NO:6 to SEQ ID NO:10, and the configuration of the double-stranded RNAs is shown in FIG. 5. All of the siRNAs were custom synthesized based on the target sequences by Dharmacon, Inc.

<Evaluation of the siRNAs>

The siRNAs were evaluated based on their capacity to inhibit hVEGF-A expression in PC-3 cells. PC-3 human prostate cancer cells (ATCC) were plated onto 35-mm dishes (2×105 cells/dish); after standing overnight, the particular siRNA was transfected (37° C., 4 hours) under serum-free conditions using a cationic lipid reagent (LipofectAMINE PLUS from Invitrogen). After transfection, 1 mL 10% FBS was added and incubation was carried out for 6 hours, after which the medium was replaced with serum-free medium containing 20 μg/mL heparin. After incubation for 16 hours, the culture supernatant was recovered and the hVEGF-A concentration was measured using a Quantikine human VEGF ELISA kit (R & D Systems). The results are shown in FIG. 6.

As shown in FIG. 6, siRNA #2 and siRNA #3 showed a high inhibitory effect on hVEGF-A, with the highest inhibitory effect being exhibited by siRNA #3.

### Example 2

The inhibition of hVEGF-A expression was monitored in this example using siRNA #3 (not modified with a stabilizing modifying group) from Example 1 and stabilized siRNA based on the same target sequence as siRNA #3. The stabilized siRNA was custom synthesized by Dharmacon, Inc., based on target sequence #3 using siSTABLE™ (Dharmacon), which is siRNA modified with a stabilizing modifying group by Dharmacon.

<siRNA Evaluation>

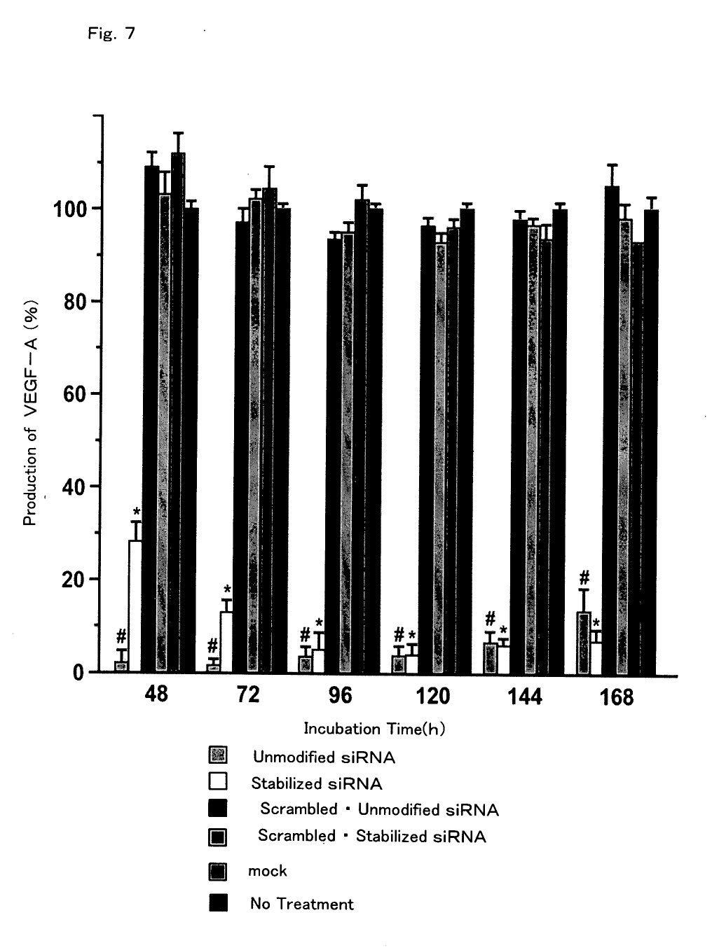

These two siRNAs were evaluated for their capacity to inhibit hVEGF-A expression in PC-3 cells. PC-3 human prostate cancer cells (ATCC) were plated onto 35-mm dishes (2×105 cells/dish); after standing overnight, the particular siRNA was transfected (37° C., 4 hours) under serum-free conditions using a cationic lipid reagent (LipofectAMINE PLUS from Invitrogen). After transfection, 1 mL 10% FBS was added and incubation was carried out for 6 hours, after which the medium was replaced with serum-free medium containing 20 μg/mL heparin and incubation was then continued. Culture supernatant was recovered at 48, 72, 96, 120, 144, and 168 hours after transfection and the hVEGF-A concentration was measured using a Quantikine human VEGF ELISA kit (R & D Systems). Using a sequence that was scrambled with respect to target sequence #3, the VEGF-A concentration was measured by the same method for the corresponding unmodified siRNA and for the corresponding stabilized siRNA that had been stabilized by Dharmacon's siSTABLE. The results are shown in FIG. 7.

As shown in FIG. 7, stabilized siRNA #3 had an approximately 70% knockdown rate at 48 hours post-transfection and its RNAi activity tended to be more strongly exhibited with elapsed time. The unmodified siRNA #3 presented a trend in which the RNAi activity gradually weakened with time; however, at 168 hours the RNAi activity of the unmodified siRNA #3 still held at about one-half that of the stabilized siRNA. The siRNAs having a scrambled siRNA #3 sequence both (both the stabilized type and the unmodified type) exhibited a complete lack of RNAi activity. The amount of hVEGF-A secretion was also unchanged for the mock (only lipid transfection reagent).

### Example 3

The optimal voltage for the electroporative transfection of siRNA into a tumor was determined in this example. The electroporative transfection method is described below. Cancer-bearing mice having a subcutaneously transplanted PC-3 tumor (xenograft) were prepared by the subcutaneous injection (24-gauge needle) of PC-3 cells (3×10⁶/site) into the inguinal region of nude mice (SLC Japan, 8-week-old males). Tumors had formed at the same site (tumor volume=50 to 80 mm³) after 3-4 weeks had elapsed after cell transplantation. These mice were used in the electroporation experiments. The tumor volume was determined using the following formula.

V=(short axis)2×(long axis)÷2

The limbs were immobilized under Nembutal anesthesia (auxiliary anesthesia: ether inhalation). A pad that had been preliminarily soaked in PBS was glued in advance on the plate-shaped electrode of plate & fork type electrodes having the shape shown in FIG. 1 (platinum rectangular plate-shaped electrode: 6.67 mm×3.87 mm, stainless fork-shaped electrode, 3 tines, length=6.67 mm, gap=1.3 mm, from NEPA GENE Co., Ltd.). A PBS solution of the stabilized siRNA was injected into the center of the tumescent subcutaneous tumor, 10 μL at each of a total of 2 locations for a total of 20 μL. The fork-shaped electrode was inserted to the underside of the subcutaneous tumor, while the plate-shaped electrode was placed on the epidermis above the tumor so as to provide a sandwiched configuration. While maintaining this state, current was passed through using a CUY21 (NEPA GENE Co., Ltd.). The energizing conditions were as follows: 3 pulse transmissions at Pon=50 msec and Poff=100 msec; then switch polarity; and the same pulse transmission 3 times, for a total of 6 pulse transmissions.

The optimal voltage for transfection of the siRNA into the tumor was determined using Cy3-labeled siSTABLE-modified siRNA #3 (custom synthesis by Dharmacon Co., Ltd.) as the siRNA. 20 μL of the Cy3-labeled stabilized siRNA (40 μM) was injected per tumor (10 μL×2 locations) and electroporation was carried out by the procedure described above. Voltages of 35, 50, 60, 70, 90, or 100 V were used to carry out current application, and the effective current value actually flowing into the tumor was recorded. The tumor was excised 24 hours later; frozen sections were prepared; and the red fluorescence from the Cy3 was observed and recorded using a confocal laser microscope. The cell nuclei were also stained with SYTOX Green (Invitrogen) reagent (green fluorescence). A group was also included and investigated in which current was not applied after the injection of 20 μL of the Cy3-labeled stabilized siRNA (40 μM) per tumor (10 μL×2 locations). Based on the results, it was found that siRNA is efficiently transfected into cells when current is applied at a voltage setting of at least 50 V but not more than 70 V.

### Example 4

The intratumoral residence of the siRNA was evaluated in this example. The same procedure as in Example 3 was carried out, except that in this case evaluation by the preparation of frozen sections from the excised tumor was carried out 5, 10, 15, and 20 days after the electroporative transfection. The Cy3-labeled material was similarly observed and recorded using a confocal laser microscope. The results showed that the Cy3-labeled stabilized siRNA persisted within the tumor for at least 20 days.

### Example 5

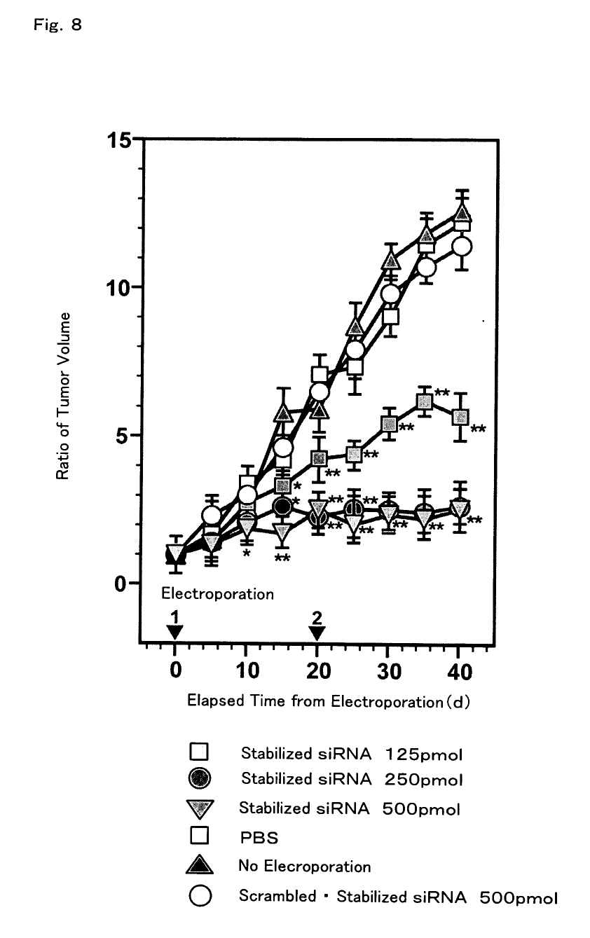

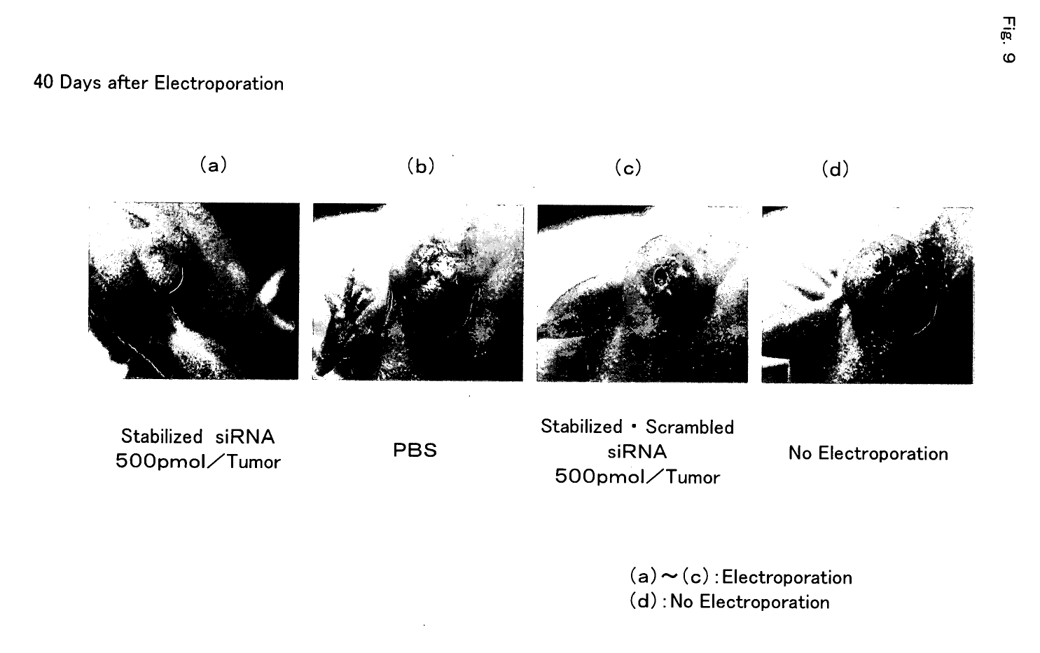

The anti-tumor effect (therapeutic effect) of siRNA by electroporation was evaluated in this example. The procedure is described in the following. Proceeding as in Example 3, a PBS solution of stabilized siRNA #3 (6.25, 12.5, or 25 μM solution) was injected (10 μL×2) into a PC-3 subcutaneous tumor (volume at start of treatment=50 to 80 mm³); the fork side of the plate & fork type electrodes was immediately inserted to the underside of the tumor; and current was applied (voltage setting=70 V in all cases). For the control, a group was also investigated in which PBS was injected into the tumor (10 μL×2) and current was applied. A group was also set up in which a PBS solution (25 μM) of scrambled-sequence stabilized siRNA was injected (10 μL×2). Another group was also set up that was not subjected to any treatment and did not receive any application of current. The day on which treatment started was designated day 0. A second treatment was carried out by entirely the same procedure on day 20. The tumor diameter was monitored until day 42. The metric for the therapeutic effect was the ratio of the tumor volume after the particular number of days had elapsed to the tumor volume on day 0 (the calculation formula is given in Example 3). Each treatment group had 5 cancer-bearing mice. The amount of siRNA used per tumor is given below. The results are shown in FIG. 8.

amount of siRNA per tumor

| | |
|---|---|
| injection of 20 μL of 6.25 μM solution | 125 pmol |
| injection of 20 μL of 12.5 μM solution | 250 pmol |
| injection of 20 μL of 25 μM solution | 500 pmol |

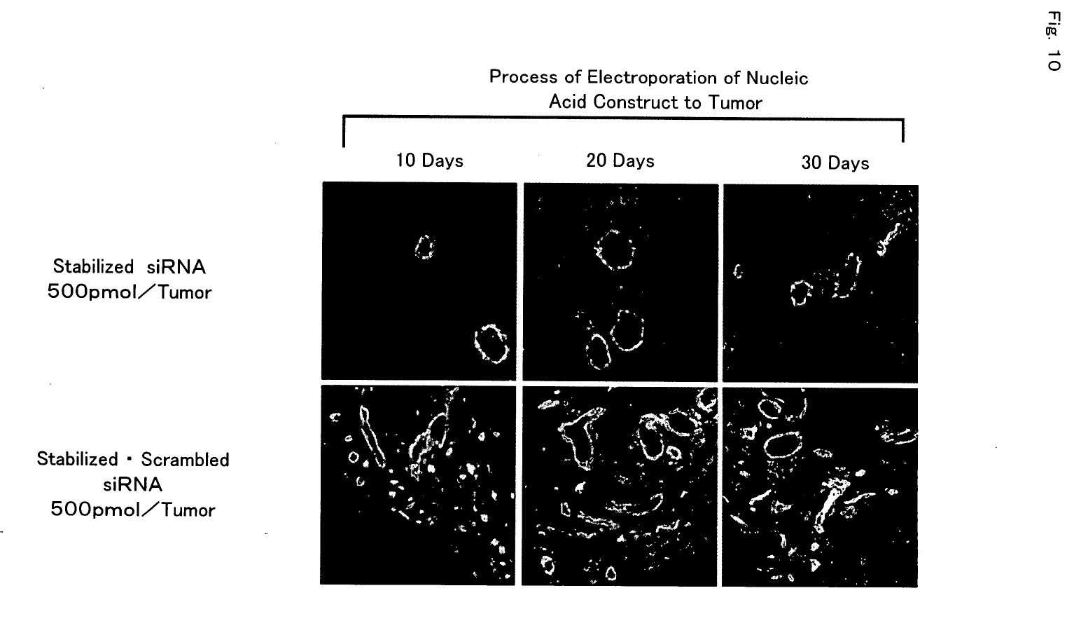

Photographs of the appearance of the tumors on the 40th day from the start of treatment are shown in FIGS. 9(a) to (d). FIG. 9(a) concerns stabilized siRNA and shows the appearance of a tumor where the amount of siRNA was 500 pmol/tumor (electroporation was carried out); FIG. 9(b) shows the appearance of a tumor for only PBS (electroporation was carried out); FIG. 9(c) concerns a scrambled stabilized siRNA having a scrambled sequence and shows the appearance of a tumor where the amount of siRNA was 500 pmol/tumor (electroporation was carried out); and FIG. 9(d) shows the appearance of a tumor that received no treatment (electroporation was not carried out).

In addition, the intratumoral microvessel density was measured by immunochemical staining using CD31 (surface antigen on vascular endothelial cells) as the marker, for the tumors that had been treated with stabilized siRNA (500 pmol/tumor) and the tumors that had received scrambled•stabilized siRNA (500 pmol/tumor). The investigation was carried on the tumors on the 10th, 20th, and 30th day from the start of treatment. The results are shown in FIG. 10.

As is shown in FIG. 8, about the same therapeutic effect (anti-tumor effect) was observed for the stabilized siRNA at 250 pmol siRNA per tumor and 500 pmol siRNA per tumor. On the other hand, the therapeutic effect was weaker when the amount of siRNA per tumor was 125 pmol. Moreover, as shown in FIGS. 8 and 9, absolutely no therapeutic effect was seen for the scrambled species. In addition, no therapeutic effect was seen for the group in which current was applied after the injection of PBS; the same was also true for the group that received neither treatment nor current application. Cancer regression due to tissue damage from the application of current was entirely absent. Based on the preceding, the electroporative delivery of siRNA was found to be a simple and rapid method that has an excellent capacity for local application and that yields a high therapeutic effect.

In a comparison with the results for siRNA delivery using atelocollagen, which the present inventors have carried out previously (Y. TAKEI et al., Cancer Research, 64, 3365-3370, May 15, 2004), the therapeutic effect for the electroporative delivery of 250 pmol siRNA per tumor in this example agreed with the therapeutic effect for 500 pmol siRNA per tumor using the atelocollagen delivery method (however, unmodified siRNA was used in the atelocollagen delivery method). Based on the preceding, it was shown that the electroporative delivery of siRNA has an efficacy comparable or superior to that of the atelocollagen delivery method.

As shown in FIG. 10, the group treated with stabilized siRNA had a clearly lower intratumoral microvessel density than the group treated with stabilized siRNA that had a scrambled sequence. This result demonstrated that the inhibitory effect on cancer growth due to this treatment was due to a mechanism of inhibition of intratumoral angiogenesis caused by an siRNA-mediated decline in the amount of hVEGF-A within the tumor.

### Example 6

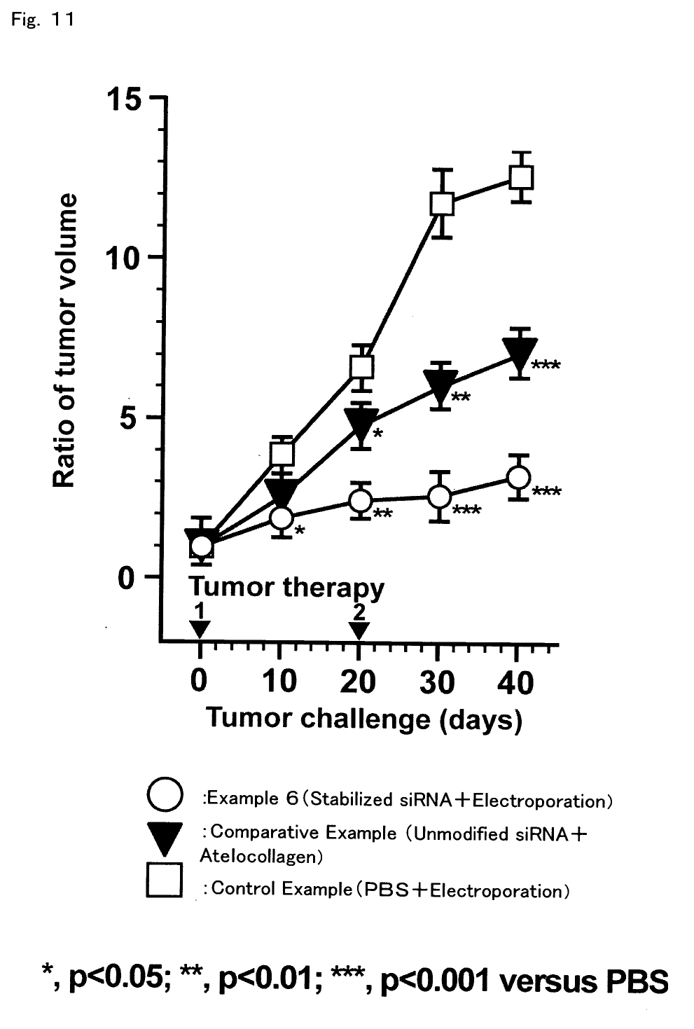

The procedure of Example 3 was followed, but in this example using a PBS solution of only stabilized siRNA #3 (12.5 μM) for the siRNA solution. The PC-3 subcutaneous tumor (volume at the start of treatment=50 to 80 mm³) was injected (10 μL×2); the fork side of the plate & fork type electrodes was immediately inserted to the underside of the tumor; the plate-shaped electrode was abutted to the epidermis so as to sandwich the subcutaneous tumor; and current was applied (voltage setting=70 V in all cases). siRNA transfection was carried out so as to provide 250 pmol per tumor. For comparison, a group was also set up in which a solution containing unmodified siRNA #3 (12.5 μM) and atelocollagen (1.75%) was injected (10 μL×2) into the subcutaneous tumor so as to provide the same amount of siRNA. For the control example, a group was set up in which PBS was injected (10 μL×2) into the subcutaneous tumor and current was applied. The day of the first treatment as designated as day 0. The treatment described above was carried out again after 20 days, and the size of each tumor was measured up to day 40. The results are shown in FIG. 11.

As shown in FIG. 11, the group electroporatively transfected with stabilized siRNA #3 by itself (the unfilled circle in the figure) gave a better therapeutic effect than the group in which unmodified siRNA #3 was transfected with atelocollagen (comparative example, filled triangle). Based on the preceding, the electroporative siRNA transfection method was shown to have a better expression of siRNA function than the atelocollagen transfection method, that is, a better inhibitory activity on gene expression and tumor-inhibiting effect were shown to occur.

### Example 7

The shape of the electrodes used for electroporation and the amount of current application used for electroporation were investigated in this example. Proceeding as in Example 3, PC-3 (human prostate cancer) cells were subcutaneously transplanted into nude mice to form tumors. Using tumor tissue where the tumor volume had reached about 50 to 80 mm³, the actual resistance value within the tumor and the actual current value within the tumor were measured when a voltage that would not damage the tissue (70 V) was applied using a pair of plate-shaped electrodes (plate electrodes (rectangular platinum plate-shaped electrodes: 6.67 mm×3.87 mm) from NEPA GENE Co., Ltd.) or a plate-shaped electrode and a needle-shaped electrode (plate & fork type electrodes having the shape shown in FIG. 1 (rectangular platinum plate-shaped electrode: 6.67 mm×3.87 mm; stainless fork-shaped electrode: 3 tines, length=6.67 mm, gap=1.3 mm; from NEPA GENE Co., Ltd.)). The pair of plate-shaped electrodes was disposed so as to sandwich the solid tumor from the sides of the tumor across the epidermis. In the case of the plate-shaped electrode and needle-shaped electrode, the needle-shaped electrode was inserted to below the bottom of the subcutaneous solid tumor while the plate-shaped electrode was disposed at the top of the solid tumor with the epidermis interposed, thus setting up a sandwich configuration. The actual values were measured on 5 solid tumors for each of these electrode geometries. The electroporation apparatus was a CUY21 (NEPA GENE Co., Ltd.), and the same energizing conditions were used for measurement of all the actual values (the energizing conditions were as follows: 3 pulse transmissions at Pon=50 msec and Poff=100 msec; then switch polarity; and the same pulse transmission 3 times, for a total of 6 pulse transmissions). The results are shown in Table 1.

TABLE 1

| | | | | | | | | | standard | t-test (vs. |
|---|---|---|---|---|---|---|---|---|---|---|
| electrode configuration | | item detected | 1 | 2 | 3 | 4 | 5 | avg. | deviation | method A) |
| A | plate and | resistance (ohm) | 929 | 1260 | 1601 | 840 | 940 | 1114 | 315 | — |
| | plate | before polarity | 0.08 | 0.09 | 0.08 | 0.05 | 0.06 | 0.072 | 0.016 | — |
| | | switch (A) | | | | | | | | |
| | | after polarity | 0.09 | 0.07 | 0.08 | 0.08 | 0.05 | 0.074 | 0.015 | — |
| | | switch (A) | | | | | | | | |
| B | plate and | resistance (ohm) | 874 | 1300 | 614 | 599 | 723 | 822 | 289 | 0.165 |
| | needle | before polarity | 0.15 | 0.09 | 0.47 | 0.23 | 0.19 | 0.226 | 0.146 | 0.047 |
| | | switch (A) | | | | | | | | |
| | | after polarity | 0.23 | 0.09 | 0.18 | 0.15 | 0.28 | 0.186 | 0.073 | 0.010 |
| | | switch (A) | | | | | | | | |

As shown in Table 1, the use of the plate-shaped electrode and needle-shaped electrode gave resistance values there were not significantly different from those for the use of the pair of plate-shaped electrodes, but significantly higher current values were measured with the former. The present inventors have developed the knowledge that the amount of current for an efficient transfection of siRNA into tumor tissue is about 0.2 A. In accordance with this knowledge, it is presumed that when plate-shaped electrodes are used a secure transfection of siRNA cannot be expected at an applied voltage of 70 V, which has a low biotoxicity for the tumor tissue. While the amount of current can be increased by increasing the applied voltage, this produces tissue toxicity and shedding and in some cases may also make it impossible to secure the safety of the organism.

In regard to the mode of disposing such a plate-shaped electrode and needle-shaped electrode, by having the needle-shaped electrode and the plate-shaped electrode face each other, the biological tissue, e.g., a solid tumor, can be securely sandwiched and can be sandwiched so as to provide a constant inter-electrode gap, regardless of whether the subcutaneous tissue is directly sandwiched by the two electrodes or is sandwiched with the needle-shaped electrode disposed subcutaneously and the plate-shaped electrode disposed with the epidermis interposed. This is presumed to enable a high current level to be easily obtained. Furthermore, since angiogenesis is active within the tumor and the vascular permeability is also high, it is thought that the current level can be readily secured when the needle-shaped electrode is inserted into the vicinity of such tissue. Based on the preceding, it can be concluded that electroporation is preferably executed using the combination of a plate-shaped electrode and a needle-shaped electrode to directly sandwich the biological tissue or to indirectly sandwich subcutaneous tissue across the epidermis in a low-invasive manner.

It is believed that the plate-shaped electrode +needle-shaped electrode combination is well suited to those instances where the target biological tissue is tumor tissue (particularly subcutaneous tumor tissue) and the target gene for the nucleic acid construct to be transfected, e.g., siRNA, is a vascular endothelial growth factor (VEGF), and that in these instances electroporation by the application of voltage using the above-described disposition mode with respect to the solid tumor is effective.

This application cites priority to Japanese patent Application No. 2005-207067 filed on 16 Jul. 2005, whose contents are incorporated herein in their entirety by reference.

### INDUSTRIAL APPLICABILITY

The present invention is useful for the production of electroporation apparatuses and for their applications.

### SEQUENCE LISTING FREE TEXT

SEQ ID NO:1 to SEQ ID NO:5: target sequence of siRNA to human VEGF-A

SEQ ID NO:6 to SEQ ID NO:10: sense sequence of siRNA

1. A method of controlling an electroporation apparatus for an animal encompassing humans and nonhuman animals, comprising a step of applying, in the presence of a nucleic acid construct capable of inhibiting the expression of a gene in the animal, voltage to electrodes of the electroporation apparatus that are disposed at biological tissue of the animal.

2. The method according to claim 1, wherein the nucleic acid construct is selected from single-stranded and double-stranded DNAs, single-stranded and double-stranded RNAs, DNA-RNA hybrids, and DNA-RNA chimeric oligonucleotides.

3. The method according to claim 1, wherein the nucleic acid construct is a nucleic acid construct capable of expressing RNA interference in the animal.

4. The method according to claim 3, wherein the nucleic acid construct is siRNA.

5. The method according to claim 1, wherein the stability of the nucleic acid construct in serum has been improved by modification.

6. The method according to claim 5, wherein the nucleic acid construct has a half-life in human serum of at least 50 hours.

7. The method according to claim 1, wherein the nucleic acid construct is a construct capable of expressing RNA interference, with a gene that promotes angiogenesis being targeted.

8. The method according to claim 7, wherein the gene is a vascular endothelial growth factor gene.

9. The method according to claim 1, wherein the biological tissue comprises a solid tumor.

10. The method according to claim 1, wherein the biological tissue is tissue present in the epidermis or subepidermis.

11. The method according to claim 1, comprising a step of supplying a biodegradable matrix material to the biological tissue or into the neighborhood thereof, prior to or after or at the same time as the application of voltage to the electrodes.

12. The method according to claim 1, wherein the voltage application step is a step in which the voltage is applied to the electrodes disposed at the biological tissue, after or while supplying the nucleic acid construct to the biological tissue or to the vicinity thereof.

13. The method according to claim 1, wherein the electrodes comprise at least one plate-shaped electrode.

14. The method according to claim 1, wherein the electrodes comprise one or two or more needle-shaped electrodes.

15. The method according to claim 14, wherein the needle-shaped electrode comprises an orifice and a hollow part through which liquid containing the nucleic acid construct can transit.

16. The method according to claim 15, wherein the electroporation apparatus comprises the plate-shaped electrode and the needle-shaped electrode disposed as counter electrodes.

17. The method according to claim 16, wherein the plate-shaped electrode is disposed on the surface of the biological tissue into which the nucleic acid construct is to be transfected and the needle-shaped electrode is disposed puncturing into this biological tissue or into the vicinity thereof.

18. The method according to claim 17, wherein the biological tissue is a subcutaneous solid tumor; the plate-shaped electrode is disposed abutting on the surface of the epidermis that covers the subcutaneous solid tumor; and the needle-shaped electrode is disposed puncturing into this biological tissue or into the vicinity thereof.

19. The method according to claim 1, wherein the voltage applied to the electrodes is at least 50 V and not more than 70 V.

20. A method of controlling an electroporation apparatus for an animal encompassing humans and nonhuman animals, comprising a step of applying, in the presence of siRNA capable of expressing RNA interference in the animal, a prescribed voltage across a needle-shaped electrode that is disposed in the lower portion of subepidermal diseased tissue in the animal and a plate-shaped electrode that is disposed on the surface of the epidermis that covers the diseased tissue.

21. The method according to claim 20, wherein the animal is a human, and the diseased tissue contains tissue for which an inhibition of progression, improvement, or treatment is possible through an inhibition of angiogenesis.

22. A method of producing a nonhuman animal, comprising a step of transfecting, by electroporation, a nucleic acid construct capable of inhibiting the expression of a gene in the nonhuman animal into cells of biological tissue of the nonhuman animal.

23. The production method according to claim 22, wherein the nucleic acid construct is a nucleic acid construct capable of expressing RNA interference, with a disease-associated gene being targeted.

24. A method of producing a nonhuman animal, comprising the steps of:
preparing a nonhuman animal that has a pathological condition, genetic mutation, or biological tissue or cell phenotype capable of manifesting as a model of a human disease; and
transfecting, by electroporation, a nucleic acid construct capable of inhibiting the expression of a gene in the nonhuman animal into cells of biological tissue of the nonhuman animal.

25. A method of identifying a therapeutic agent, comprising the steps of:
preparing a nonhuman animal that has a pathological condition, genetic mutation, or biological tissue or cell phenotype capable of manifesting as a model of a human disease;
transfecting, by electroporation, a nucleic acid construct capable of inhibiting the expression of a gene in the nonhuman animal into cells of biological tissue of the nonhuman animal; and
analyzing the pathological condition or the biological tissue or cell phenotype of the animal model into which the nucleic acid construct has been transfected.

26. A method of identifying a therapeutic agent, comprising the steps of:
transfecting, by electroporation, a nucleic acid construct capable of inhibiting the expression of a gene in a nonhuman animal into cells of biological tissue of the nonhuman animal to form a pathological condition or biological tissue or cell phenotype for a human disease in at least a portion of the nonhuman animal; and
administering a compound to the nonhuman animal and analyzing the pathological condition or biological tissue or cell phenotype.

27. A method of identifying a target compound for drug discovery, comprising the steps of:
transfecting, by electroporation, a nucleic acid construct that is capable of inhibiting gene expression in a nonhuman animal, with a disease-associated gene in the nonhuman animal being targeted, into cells of biological tissue of the nonhuman animal; and
analyzing the phenotype of the aforementioned cells or biological tissue into which the nucleic acid construct has been transfected.


SEQUENCE LISTING (10 sequences)

SEQ ID NO: 1 - DNA, 19 bp, Artificial Sequence
  Synthetic Construct target sequence of siRNA to hVEGF-A
tggatgtctatcagcgcag

SEQ ID NO: 2 - DNA, 19 bp, Artificial Sequence
  Synthetic Construct target sequence of siRNA to hVEGF-A
gctactgccatccaatcga

SEQ ID NO: 3 - DNA, 19 bp, Artificial Sequence
  Synthetic Construct target sequence of siRNA to hVEGF-A
ggagtaccctgatgagatc

SEQ ID NO: 4 - DNA, 19 bp, Artificial Sequence
  Synthetic Construct target sequence of siRNA to hVEGF-A
ctgaggagtccaacatcac

SEQ ID NO: 5 - DNA, 19 bp, Artificial Sequence
  Synthetic Construct target sequence of siRNA to hVEGF-A
ccaaggccagcacatagga

SEQ ID NO: 6 - RNA, 19 nt, Artificial Sequence
  Synthetic Construct sense sequence of siRNA to hVEGF-A
uggaugucuaucagcgcag

SEQ ID NO: 7 - RNA, 19 nt, Artificial Sequence
  Synthetic Construct sense sequence of siRNA to hVEGF-A
gcuacugccauccaaucga

SEQ ID NO: 8 - RNA, 19 nt, Artificial Sequence
  Synthetic Construct sense sequence of siRNA to hVEGF-A
ggaguacccugaugagauc

SEQ ID NO: 9 - RNA, 19 nt, Artificial Sequence
  Synthetic Construct sense sequence of siRNA to hVEGF-A
cugaggaguccaacaucac

SEQ ID NO: 10 - RNA, 19 nt, Artificial Sequence
  Synthetic Construct sense sequence of siRNA to hVEGF-A
ccaaggccagcacauagga